SEQUENCE LISTING (11 sequences)

SEQ ID NO: 1 - protein, 270 aa, Synthetic construct
MLQACKMEGFPLVPPPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 2 - protein, 358 aa, Synthetic construct
MESADFYEAEPRPPMSSHLQSPPHAPSSAAFGFPRGAGPAQPPAPPAAPEPLGGICEHETSIDISAYIDPAAFNDEFLADLFQHSRQQEKAKAAVGPTGGGGGGDFDYPGAPAGPGGAVMPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 3 - protein, 452 aa, Synthetic construct
MDGTIKEALSVVSDDQSLFDSAYGAAAHLPKADMTASGSPDYGQPHKINPLPPQQEWINQPVRVNVKREYDHMNGSRESPVDCSVSKCSKLVGGGESNPMNYNSYMDEKNGPPPPNMTTNERRVIVPADPTLWTQEHVRQWLEWAIKEYSLMEIDTSFFQNMDGKELCKMNKEDFLRATTLYNTEVLLSHLSYLRESSLLAYNTTSHTDQSSRLSVKEDPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 4 - protein, 473 aa, Synthetic construct
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 5 - protein, 345 aa, Synthetic construct
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 6 - protein, 426 aa, Synthetic construct
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVQVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 7 - DNA, 52 bp, Synthetic construct
  source 1..52: mol_type = other DNA
ggagttcagacgtgtgctcttccgatctcttttccaagcacctgctacatag

SEQ ID NO: 8 - DNA, 70 bp, Synthetic construct
  source 1..70: mol_type = other DNA
aatgatacggcgaccaccgagatctacacaactcgctacactctttccctacacgacgctcttccgatct

SEQ ID NO: 9 - DNA, 66 bp, Synthetic construct
  source 1..66: mol_type = other DNA
caagcagaagacggcatacgagattcgccttagtgactggagttcagacgtgtgctcttccgatct

SEQ ID NO: 10 - DNA, 20 bp, Synthetic construct
  source 1..20: mol_type = other DNA
aatgatacggcgaccaccga

SEQ ID NO: 11 - DNA, 21 bp, Synthetic construct
  source 1..21: mol_type = other DNA
caagcagaagacggcatacga

(19) United States
(12) Patent Application Publication
(10) Pub. No.: US 2025/0263655 A1
(43) Pub. Date: Aug. 21, 2025

(54) METHODS AND COMPOSITIONS FOR PRODUCING MICROGLIA

(71) Applicant: President and Fellows of Harvard College, Cambridge, MA (US)

(72) Inventors: Songlei Liu, Cambridge, MA (US); George M. Church, Cambridge, MA (US)

(73) Assignee: President and Fellows of Harvard College, Cambridge, MA (US)

(21) Appl. No.: 18/858,608

(22) PCT Filed: Apr. 20, 2023
(86) PCT No.: PCT/US2023/066023

§ 371 Date: Oct. 21, 2024

Related U.S. Application Data
(60) Provisional application No. 63/333,573, filed on Apr. 22, 2022.

(51) Int. Cl. C12N 5/079; C12N 5/074

(52) U.S. Cl. CPC C12N 5/0622; C12N 5/0696; C12N 2501/999; C12N 2510/00

(57) ABSTRACT

Provided herein are methods and compositions for differentiating induced pluripotent stem cells into microglia-like cells by overexpressing transcription factors such as SPI1, CEBPA, FLU, MEF2C, CEBPB, and/or IRF8.

### CROSS-REFERENCE TO RELATED APPLICATIONS

This application claims the benefit under 35 U.S.C. § 119(e) of U.S. Provisional Application No. 63/333,573, filed Apr. 22, 2022, which is hereby incorporated by reference in its entirety.

### GOVERNMENT LICENSE RIGHTS

This invention was made with government support under HG008525 awarded by National Institutes of Health (NIH). The government has certain rights in the invention.

### REFERENCE TO AN ELECTRONIC SEQUENCE LISTING

The contents of the electronic sequence listing (H049870762WO00-SEQ-KVC.xml; Size: 13,069 bytes; and Date of Creation: Apr. 19, 2023) is herein incorporated by reference in its entirety.

### BACKGROUND

Microglia are the resident immune cells of the brain, which originated from erythro-myeloid progenitors (EMPs) in the yolk sac. They play important and diverse roles in brain development and maintaining homeostasis. Recent studies have demonstrated the link between neuroinflammation and neurodegenerative disease, such as Alzheimer's Disease (AD), and along these lines, microglia have been shown to be an important cell type in AD and other neurodegenerative diseases. Functional studies to define therapeutics targeting human microglia have been greatly hindered by the limited availability of human brain biopsies. The supply issue cannot simply be mitigated by using murine models, because differences between human and mouse microglia limit the transferability of knowledge. Producing human microglia-like cells from hiPSCs might fill this gap. Several studies have accomplished this goal through a process of embryoid body formation, growth factor treatment, and, in some cases, co-culturing with neurons. These protocols draw inspiration from the natural developmental stages of microglia and have timelines ranging from 30-74 days.

### SUMMARY

As the effects of extrinsic factors on cell fate are frequently mediated by TFs, direct manipulations of TF expression could differentiate hiPSCs to microglia in a shorter timeframe. The ability to differentiate stem cells into human cell types is a crucial tool to define basic mechanisms and therapeutics, especially for cell types not routinely accessible by biopsies. But while engineered expression of transcription factors (TFs) identified through TF screens has been found to rapidly and efficiently produce some cell types, generation of types that require complex combinations of TFs has been elusive. Here, an iterative, pooled single-cell TF screening method was developed, which improves the identification of effective TF combinations using the generation of human microglia-like cells as a testbed. Two iterations identified a combination of SPI1, CEBPA, FLI1, MEF2C, CEBPB, and IRF8 as sufficient to differentiate human iPSC into microglia-like cells in about four (4) days, for example. Characterization of TF-induced microglia demonstrated molecular and functional similarity to primary microglia. The use of single-cell atlas reference datasets was explored to confirm identified TFs and how combining single-cell TF perturbation and gene expression data can enable the construction of causal gene regulatory networks. What will be needed to fashion these methods into a generalized integrated pipeline, further ideas for enhancement, and possible applications are described herein.

Some aspects provide a pluripotent stem cell (PSC) comprising: one or more engineered polynucleotide comprising an open reading frame encoding a SPI1 protein, a FLI1 protein, and a CEBPA protein.

In some embodiments, the one or more engineered polynucleotide comprises a first polycistronic polynucleotide that comprises a first open reading frame encoding the SPI1 protein, a second open reading frame encoding the FLI1 protein, and a third open reading frame encoding the CEBPA protein.

In some embodiments, the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the FLI1 protein, and a third open reading frame encoding the CEBPA protein.

In some embodiments, the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the CEBPA protein, and a third open reading frame encoding the FLI1 protein.

In some embodiments, the one or more engineered polynucleotide comprises an open reading frame encoding one or more of a MEF2C protein, a CEBPB protein, and a IRF8 protein.

In some embodiments, the one or more engineered polynucleotide comprises an open reading frame encoding two or more of a MEF2C protein, a CEBPB protein, and a IRF8 protein.

In some embodiments, the one or more engineered polynucleotide comprises an open reading frame encoding a MEF2C protein, a CEBPB protein, and a IRF8 protein.

In some embodiments, the one or more engineered polynucleotide comprises a second polycistronic polynucleotide that comprises the following open reading frames, ordered in the 5′ to 3′ direction of the second polycistronic polynucleotide: a first open reading frame encoding the MEF2C protein, a second open reading frame encoding the CEBPB protein, and a third open reading frame encoding the IRF8 protein.

In some embodiments, the one or more engineered polynucleotide comprises a second polycistronic polynucleotide that comprises the following open reading frames, ordered in the 5′ to 3′ direction of the second polycistronic polynucleotide: a first open reading frame encoding the MEF2C protein, a second open reading frame encoding the IRF8 protein, and a third open reading frame encoding the CEBPB protein.

In some embodiments, the one or more engineered polynucleotide comprises: a first polycistronic polynucleotide that comprises, optionally 5′ to 3′, a first open reading frame encoding the SPI1 protein, a second open reading frame encoding the CEBPA protein, and a third open reading frame encoding the FLI1 protein; and a second polycistronic polynucleotide that comprises, optionally 5′ to 3′, a first open reading frame encoding the MEF2C protein, a second open reading frame encoding the CEBPB protein, and a third open reading frame encoding the IRF8 protein.

In some embodiments, one or more of the open reading frames of the one or more engineered polynucleotide is operably linked to a heterologous promoter.

In some embodiments, the heterologous promoter is an inducible promoter.

In some embodiments, the inducible promoter is a chemically-inducible promoter.

Other aspects provide pluripotent stem cell (PSC) comprising: a SPI1 protein, a FLI1 protein, and a CEBPA protein, wherein the proteins are overexpressed.

In some embodiments, the PSC further comprises one or more of a MEF2C protein, a CEBPB protein, and a IRF8 protein, wherein the one or more of the MEF2C protein, the CEBPB protein, and the IRF8 protein is overexpressed.

In some embodiments, the PSC further comprises two or more of a MEF2C protein, a CEBPB protein, and a IRF8 protein, wherein the two or more of the MEF2C protein, the CEBPB protein, and the IRF8 protein are overexpressed.

In some embodiments, the PSC further comprises a MEF2C protein, a CEBPB protein, and a IRF8 protein, wherein the MEF2C protein, the CEBPB protein, and the IRF8 protein are overexpressed.

In some embodiments, the PSC is a human PSC.

In some embodiments, the PSC is an induced PSC (iPSC).

Some aspects provide composition comprising: a population of the PSC of any one of the preceding paragraphs.

Other aspects provide method, comprising: culturing, in culture media, a population of pluripotent stem cells (PSCs) to produce an expanded population of PSCs; and expressing in PSCs of the expanded population a SPI1 protein, a CEBPA protein, and a FLI1 protein, to produce a population of microglia-like cells.

In some embodiments, the PSCs of the expanded population comprise one or more engineered polynucleotide comprising an open reading frame encoding a SPI1 protein, a FLI1 protein, and a CEBPA protein.

In some embodiments, the one or more engineered polynucleotide comprises a first polycistronic polynucleotide that comprises a first open reading frame encoding the SPI1 protein, a second open reading frame encoding the FLI1 protein, and a third open reading frame encoding the CEBPA protein.

In some embodiments, the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the FLI1 protein, and a third open reading frame encoding the CEBPA protein.

In some embodiments, the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the CEBPA protein, and a third open reading frame encoding the FLI1 protein.

In some embodiments, the method, further comprises expressing in PSCs of the expanded population one or more of a MEF2C protein, a CEBPB protein, and a IRF8 protein.

In some embodiments, the one or more engineered polynucleotide further comprises an open reading frame encoding one or more of a MEF2C protein, a CEBPB protein, and a IRF8 protein.

In some embodiments, the method further comprising expresses in PSCs of the expanded population two or more of a MEF2C protein, a CEBPB protein, and a IRF8 protein.

In some embodiments, the one or more engineered polynucleotide further comprises an open reading frame encoding two or more of a MEF2C protein, a CEBPB protein, and a IRF8 protein.

In some embodiments, the method further comprises expressing in PSCs of the expanded population a MEF2C protein, a CEBPB protein, and a IRF8 protein.

In some embodiments, the one or more engineered polynucleotide further comprises an open reading frame encoding a MEF2C protein, a CEBPB protein, and a IRF8 protein.

In some embodiments, the one or more engineered polynucleotide further comprises a second polycistronic polynucleotide that comprises the following open reading frames, ordered in the 5′ to 3′ direction of the second polycistronic polynucleotide: a first open reading frame encoding the MEF2C protein, a second open reading frame encoding the CEBPB protein, and a third open reading frame encoding the IRF8 protein.

In some embodiments, the one or more engineered polynucleotide further comprises a second polycistronic polynucleotide that comprises the following open reading frames, ordered in the 5′ to 3′ direction of the second polycistronic polynucleotide: a first open reading frame encoding the MEF2C protein, a second open reading frame encoding the IRF8 protein, and a third open reading frame encoding the CEBPB protein.

In some embodiments, the PSCs of the expanded population comprise:
a first polycistronic polynucleotide that comprises, optionally 5′ to 3′, a first open reading frame encoding the SPI1 protein, a second open reading frame encoding the CEBPA protein, and a third open reading frame encoding the FLI1 protein; and a second polycistronic polynucleotide that comprises, optionally 5′ to 3′, a first open reading frame encoding the MEF2C protein, a second open reading frame encoding the CEBPB protein, and a third open reading frame encoding the IRF8 protein.

In some embodiments, one or more of the open reading frames of the one or more engineered polynucleotide is operably linked to a heterologous promoter.

In some embodiments, the heterologous promoter is an inducible promoter.

In some embodiments, the population of PSCs comprises 1×10²-1×10⁷ PSCs.

In some embodiments, the population of PSCs is cultured for about 2-5 days.

In some embodiments, the population of PSCs is cultured for about 4 days.

In some embodiments, microglia-like cells of the population of microglia-like cells are CD11b⁺, CX3CR1⁺, ITGAM⁺, P2RY12⁺, TMEM119⁺, and/or TREM2⁺.

In some embodiments, the microglia-like cells of the population of microglia-like cells are TRA-1-60⁻ and/or POU5F1⁻.

Some aspects provide a method comprising: (a) delivering to pluripotent stem cells (PSCs) one or more engineered polynucleotide comprising an inducible promoter operably linked to one or more open reading frame encoding a SPI1 protein, a CEBPA protein, and a FLI1 protein, optionally wherein the one or more engineered polynucleotide further comprises an inducible promoter operably linked to one or more open reading frame encoding a MEF2C protein, a CEBPB protein, and/or a IRF8 protein; and (b) culturing the PSCs of the expanded population in induction media comprising an inducing agent to produce CD11b⁺, CX3CR1⁺, ITGAM⁺, P2RY12⁺, TMEM119⁺, TREM2⁺, TRA-1-60⁻ and/or POU5F1⁻ microglia-like cells.

Other aspects provide a method comprising: (a) delivering to pluripotent stem cells (PSCs) one or more engineered polynucleotide comprising an inducible promoter operably linked to one or more open reading frame encoding a SPI1 protein, a CEBPA protein, and a FLI1 protein, optionally wherein the one or more engineered polynucleotide further comprises an inducible promoter operably linked to one or more open reading frame encoding a MEF2C protein, a CEBPB protein, and/or a IRF8 protein; (b) seeding the PSCs in feeder-free, serum-free culture media and optionally culturing the PSCs for about 1 to about 24 hours; and (c) culturing the PSCs of (b) in induction media comprising an inducing agent to produce CD11b⁺, CX3CR1⁺, ITGAM⁺, P2RY12⁺, TMEM119⁺, TREM2⁺, TRA-1-60⁻ and/or POU5F1⁻ microglia-like cells.

In some embodiments, the method further comprises delivering to PSCs (i) a first polycistronic polynucleotide comprising a first inducible promoter operably linked to an open reading frame encoding SPI1, an open reading frame encoding CEBPA, and an open reading frame encoding FLI1, and (ii) a second polycistronic polynucleotide comprising a second inducible promoter operably linked to an open reading frame encoding MEF2C, open reading frame encoding CEBPB, and an open reading frame encoding IRF8.

In some embodiments, the first and/or second polycistronic polynucleotide is a transposon and the delivering further comprises delivering a transposase to the PSCs.

In some embodiments, the first and/or second inducible promoter is a chemically-inducible promoter, optionally a doxycycline-inducible promoter.

In some embodiments, the feeder-free, serum-free culture media of (b) comprises a solubilized basement membrane preparation extracted from the Engelbreth-Holm-Swarm (EHS) mouse sarcoma.

In some embodiments, the solubilized basement membrane preparation comprises extracellular matrix (ECM) proteins and growth factors.

In some embodiments, the ECM proteins are selected from Laminin, Collagen IV, heparan sulfate proteoglycans, and entactin/nidogen.

In some embodiments, the feeder-free, serum-free culture media of further comprises growth factors selected from recombinant human basic fibroblast growth factor (rh bFGF) and recombinant human transforming growth factor β (rh TGFβ).

In some embodiments, the PSCs of the expanded population of (c) are cultured at a density of about 20,000 cells/cm² to about 60,000 cells/cm².

In some embodiments, the feeder-free, serum-free culture media of (b) further comprises a small molecule ROCK inhibitor.

In some embodiments, the inducing agent comprises doxycycline.

In some embodiments, the culturing the PSCs is an induction media is for about 72 to about 96 hours.

Some aspects provide microglia-like cell produced by the method of any one of the preceding paragraphs.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1I. The first iteration of TF screen identified initial microglia differentiation factors. (FIG. 1A) Workflow of the first pooled TF screen. (FIG. 1B) Flow cytometry analysis of stem cell (TRA-1-60) and microglia (P2RY12, CD11b, CX3CR1) proteins in the PGP1+40 TF pool before and after Dox induction. (FIG. 1C) Cells with low TRA-1-60 expression in the Dox+ group were sorted for scRNA-seq. (FIG. 1D) Clustering of two independently transfected and differentiated PGP1 iPSC pools. (FIG. 1E) Expression of microglia (ITGAM, CX3CR1, TMEM119, P2RY12, TREM2) and spiked-in stem cell (POU5F1) gene in scRNA-seq. (FIG. 1F) Primer designs for co-amplification of TF and cell barcodes in 10× Genomics 3′ workflow. (FIG. 1G) Number of TFs per cell counted from normalized and binarized TF expression matrix. (FIG. 1H) Ranking of the 40 TFs after Wilcoxon rank sum test with the two tested groups being with or without microglia gene expression. Stars highlight top ranking TFs. (FIG. 11) Flow cytometry validation of top-ranking TFs for inducing microglia protein expression.

FIGS. 2A-2J. The second iteration based on the first identified additional TFs for microglia differentiation. (FIG. 2A) Workflow of the second pooled TF screen. (FIG. 2B) Polycistronic cassette design for performing dual-drug selection to achieve 3+X TF screen. (FIG. 2C) Normalized mRNA expression from the polycistronic cassette (SPI1, FLI1, CEBPA) and stem cells (POU5F1). (FIG. 2dD) TF barcode counting enabled identification of stem cells (“No TF BC”), MG3.1-SFC and cells with additional TFs (“SFC+X”). (FIG. 2e) Example histograms of TF barcode raw counts in single cells. (FIG. 2F) Number of TFs per cell counted from normalized and binarized TF expression matrix. (FIG. 2G) Ranking of the 42 TFs after Wilcoxon rank sum test with the two tested groups being with or without microglia gene expression. Stars highlight top ranking TFs. Grey highlights the SFC polycistronic cassette. (FIG. 2H) Flow cytometry validation of top-ranking TFs for improving microglia protein expression. (FIG. 2I) Polycistronic cassettes design for varying TF orders. (FIG. 2J) Flow cytometry analysis of different arrangements of the six-TF recipe in comparison with MG3.1-SFC.

FIGS. 3A-3J. TFiMGLs differentiate quickly, are phagocytic and responsive to ADP stimulation. (FIG. 3A) Expression of the six induced TFs over time measured by bulk RNA-seq. (FIG. 3B) Expression of stem cell (POU5F1) and microglia (ITGAM, CX3CR1, TMEM119, P2RY12, TREM2) genes over time measured by bulk RNA-seq. (FIG. 3C) PCA plot for the transcriptome of TFiMGLs (MG6.4) over time. (FIG. 3D) Immunofluorescence of stem cell (OCT4), Dox-induced (PU.1), and microglia (CD11b, P2RY12, CX3CR1) proteins on day 4. Scale bar: 20 μm. (FIG. 3E) Flow cytometry quantification of microglia protein expression on day 4 (n=3). (FIG. 3F) Flow cytometry analysis of the uptake of pHrodo-labeled S. aureus Bioparticles over time (n=3). (FIG. 3G) Microscopy analysis of particle uptake combined with microglia surface protein staining. (FIG. 3H, FIG. 3I, FIG. 3J) Calcium imaging with Fluo-4 after stimulation with 150 μM ADP and peak quantification.

FIGS. 4A-4E. Transcriptome analysis of TFiMGLs on different days and under disease relevant stimulations. (FIG. 4A) PCA of bulk RNA-seq data from multiple sources containing primary microglia. MG: Microglia; DC: dendritic cell; HPC: hematopoietic progenitor; iMGL: growth factor-induced microglia-like cell; Mono: monocyte; iPS: induced pluripotent stem cell. (FIG. 4B) GSEA of MG6.4 versus iPS using two microglia marker gene sets from MSigDB: M40168 and M39077. (FIG. 4C) PCA of MG6.4's transcriptome after 24 hours treatment with IFNγ, fAβ, or TDP43. (FIG. 4D, FIG. 4E) Pathway analysis of significantly differentially expressed genes after treatment with IFNγ or TDP43.

### DETAILED DESCRIPTION

The ability to produce human cell types from stem cells provides crucial tools for basic research and therapeutics development, especially when the desired cell types are not easily available from human biopsy. Broader and deeper developmental biology knowledge accumulated over years have fueled the discovery and application of a number of protocols for creating specific cell types from iPSCs, some of which already entered clinical trials for treating devastating diseases like age-related macular degeneration and type 1 diabetes, while others are routinely used in laboratories for studying disease mechanisms and testing drugs. Recent global efforts on building the HCA not only added more knowledge about human development and diseases, but also presented numerous well-defined targets for cell fate engineering. Combined with the development of genetic library construction, high-throughput screening and sequencing technologies, there has never been a better time to investigate how to engineer cell fate in a systematic and multiplexed fashion.

The data provided herein demonstrated the feasibility of using iterative genetic library screen and high-throughput scRNA-seq for cell fate engineering. Using microglia differentiation, which previously did not have a TF-driven protocol, two full iterations of design-screen-validate were performed and SPI1, CEBPA, FLI1, MEF2C, CEBPB, and IRF8 identified as a recipe for driving microglia differentiation from hiPSCs within about four days, for example. Characterizations of these transcription factor-induced microglia-like cells (TFiMGLs) indicated that they differentiated quickly within about 4 days and possessed transcriptomic and functional resemblance to primary human microglia.

### Microglia-Like Cells

Some aspects of the present disclosure provide microglia-like cells and methods of producing such cells. Microglia, the innate immune cells in the central nervous system (CNS), have two primary roles in the brain that are common to myelomonocytic innate immune cells in all mammalian organs: they fight off and phagocytize viruses, bacteria, and other foreign invaders, and they remove cellular debris to facilitate wound repair. Innate immune cells are sparsely distributed in the liver, lung, and skin. In contrast, microglia occupy all regions of the CNS in a tile-like pattern. Because the entire adult mammalian brain is occupied by microglial cells, it is evident that they have functions that are specific to the brain. Microglial cells originate from a unique stem cell in the yolk sac. Microglial progenitors colonize the brain during early fetal development and initially have ameboid shapes. To ensure that appropriate neuronal connections are made during brain development, the CNS overproduces neurons and developing neurons compete for connections. Neurons that lose this competition die; microglia do not kill these neurons, but they recognize their dysfunction or degeneration and remove them.

As the prenatal brain develops, individual microglia establish microdomains and symmetrically extend processes. A cell autonomous contact inhibition helps establish and maintain the CNS network of microglia cells. Ramified microglia constitute 5% to 20% of the glial cells in the adult CNS. They are more abundant in gray than in white matter, and phylogenetically newer regions of the CNS (cerebral cortex, hippocampus) have more microglia than do older regions (cerebellum, brainstem). Regional variations in the number and shape of microglia suggest that microglial distribution and morphology are regulated by local environments and that microglia play a role in tissue homeostasis. Although many aspects of this homeostasis remain to be elucidated, microglia respond quickly and dramatically to all forms of brain pathology.

There are two main functional aspects of microglia: immune defense and CNS maintenance. As immune cells, they act as sentinels, detecting the first signs of pathogenic invasion or tissue damage in this delicate immune-privileged site that is actively protected by the brain blood barrier. Under the inflammatory conditions of an active immune response however, microglia must also moderate the potential damage to the CNS and support tissue repair and remodeling. Perhaps unsurprisingly, dysregulated microglial activation and microglia-induced inflammation is observed in virtually all brain pathologies; emerging evidence suggests that microglia exert direct effects on neurons, contributing to disease progression.

Microglial cells express a number of different biomarkers that can be used to distinguish microglia and microglia-like cells from other cell types. For example, microglia cells are typically positive for CD11b, CX3CR1, ITGAM, P2RY12, TMEM119, and/or TREM2 (i.e., they express CD11b, CX3CR1, ITGAM, P2RY12, TMEM119, and/or TREM2). Microglia cells are typically negative for TRA-1-60 and/or POU5F1 (i.e., they do not express detectable levels of TRA-1-60 and/or POU5F1). Thus, in some embodiments, the microglia-like cells produced by the methods provided herein are CD11b⁺, CX3CR1⁺, ITGAM⁺, P2RY12⁺, TMEM119⁺, and/or TREM2⁺ microglia-like cells and/or TRA-1-60⁻ and/or POU5F1⁻ microglia-like cells (i.e., cells that express CD11b, CX3CR1, ITGAM, P2RY12, TMEM119, and/or TREM2 protein but do not express detectable levels of TRA-1-60 and/or POU5F1 protein).

There are other characteristics of microglia-like cells that distinguish them from non-microglia-like cells including, but not limited to, secretion of cytokines (TNFα, IL-6, IL-10) upon stimulation (IFNγ, LPS), phagocytosis of disease (bacterial particle, beta amyloid) or other brain-related (synaptosomes) agents, and response to damage-associated molecular patterns (DAMPs) and pathogen-associated molecular patterns (PAMPs), signals that are released from damaged or dying cells (i.e. ADP) or molecular motifs conserved within microbes.

In some embodiments, at least 50% of the cells of a population of microglia-like cells is CD11b⁺. For example, at least 55%, at least 60%, at least 65%, at least 70%, at least 75%, or at least 80% of the cells of a population of microglia-like cells may be CD11b⁺. In some embodiments, about 50% to about 80% of the cells of a population of microglia-like cells is CD11b⁺. For example, about 50% to about 70%, about 60% to about 80%, or about 60% to about 70% of the cells of a population of microglia-like cells may be CD11b⁺.

In some embodiments, at least 70% of the cells of a population of microglia-like cells is P2RY12⁺. For example, at least 70%, at least 75%, at least 80%, at least 85%, or at least 90% of the cells of a population of microglia-like cells may be P2RY12⁺. In some embodiments, about 80% to about 95% of the cells of a population of microglia-like cells is P2RY12⁺. For example, about 70% to about 95%, about 75% to about 95%, or about 80% to about 95% of the cells of a population of microglia-like cells may be P2RY12⁺.

In some embodiments, at least 5% of the cells of a population of microglia-like cells is CX3CR1+. For example, at least 5%, at least 10%, or at least 15% of the cells of a population of microglia-like cells may be CX3CR1⁺. In some embodiments, about 5% to about 25% of the cells of a population of microglia-like cells is CX3CR1⁺. For example, about 5% to about 20%, about 10% to about 20%, or about 15% to about 20% of the cells of a population of microglia-like cells may be CX3CR1⁺.

### Pluripotent Stem Cells

The microglia-like cells provided herein are differentiated from pluripotent stem cells. Pluripotent stem cells are cells that have the capacity to self-renew by dividing, and to develop into the three primary germ cell layers of the early embryo (e.g., ectoderm, endoderm, and mesoderm), and therefore into all cells of the adult body, but not extra-embryonic tissues such as the placenta (Shi et al. 2017).

Non-limiting examples of pluripotent stem cells include induced pluripotent cell (iPSCs), “true” embryonic stem cell (ESCs) derived from embryos, embryonic stem cells made by somatic cell nuclear transfer (ntESCs), and embryonic stem cells from unfertilized eggs (parthenogenesis embryonic stem cells, or pESCs). In some embodiments, a pluripotent cell is a human pluripotent cell.

In some embodiments, a pluripotent stem cell is an embryonic stem cell, such as a human embryonic stem cell. “Embryonic stem cell” is a general term for pluripotent stem cells that are made using embryos or eggs, rather than for cells genetically reprogrammed from the body. As used herein, “ESCs” encompass true ESCs, ntESCs, and pESCs.

In other embodiments, a pluripotent stem cell is an induced pluripotent stem cell, such as a human induced pluripotent stem cell. iPSCs may be derived from skin or blood cells that have been reprogrammed back into an embryonic-like pluripotent state that enables the development of an unlimited source of any type of human cell.

Some aspects of the present disclosure provide a PSC comprising: a protein selected from SPI1, CEBPA, FLI1, MEF2C, CEBPB, and IRF8, wherein the protein is expressed or overexpressed. In some embodiments, the protein is expressed at a level that is at least 5%, at least 10%, at least 15%, at least 20%, at least 25%, at least 50%, or at least 100% higher than a control level. In some embodiments, a control level is an endogenous level of the protein, for example in a naturally-occurring pluripotent stem cell. In some embodiments, a PSC comprises SPI1. In some embodiments, a PSC expresses or overexpresses SPI1. In some embodiments, a PSC comprises CEBPA. In some embodiments, a PSC expresses or overexpresses CEBPA. In some embodiments, a PSC comprises FLI1. In some embodiments, a PSC expresses or overexpresses FLI1. In some embodiments, a PSC comprises MEF2C. In some embodiments, a PSC expresses or overexpresses MEF2C. In some embodiments, a PSC comprises CEBPB. In some embodiments, a PSC expresses or overexpresses CEBPB. In some embodiments, a PSC comprises IRF8. In some embodiments, a PSC expresses or overexpresses IRF8.

Data provided herein shows that combinatorial expression of SPI1, FLI1, and CEBPA results in a population of cells in which 37% are CD11b+ and 6% are P2RY12+. Additional data provided herein shows that combinatorial expression of SPI1, FLI1, CEBPA, MEF2C, CEBPB, and IRF8 results in a population of cells in which 66% are CD11b+, 93% are P2RY12+ and 16% are CX3CR1+ at day 4 following induced expression the transcription factors. In some embodiments, a PSC comprises SPI1, FLI1, and CEBPA. In some embodiments, a PSC expresses or overexpresses SPI1, FLI1, and CEBPA. In some embodiments, a PSC comprises SPI1, FLI1, CEBPA and one or more of MEF2C, CEBPB, and IRF8. In some embodiments, a PSC comprises SPI1, FLI1, CEBPA and two or more of MEF2C, CEBPB, and IRF8. In some embodiments, a PSC comprises SPI1, FLI1, CEBPA, MEF2C, CEBPB, and IRF8. In some embodiments, a PSC expresses or overexpresses SPI1, FLI1, CEBPA and one or more of MEF2C, CEBPB, and IRF8. In some embodiments, a PSC expresses or overexpresses SPI1, FLI1, CEBPA and two or more of MEF2C, CEBPB, and IRF8. In some embodiments, a PSC expresses or overexpresses SPI1, FLI1, CEBPA, MEF2C, CEBPB, and IRF8.

### Transcription Factors

The microglia-like cells provided herein are differentiated from pluripotent stem cells, in some embodiments, by expressing one or more (e.g., 2, 3, 4, 5, 6, 7, 8, or 9) transcription factors (i.e., a protein that controls the rate of transcription). Differentiation is the process by which an uncommitted cell or a partially committed cell commits to a specialized cell fate. Aspects of the present disclosure relate to the differentiation of uncommitted pluripotent stem cells into a microglia-like cell fate.

In some embodiments, the transcription factors are selected from SPI1, CEBPA, FLI1, MEF2C, CEBPB, and IRF8. In some embodiments, pluripotent stem cells, such as hPSCs or hiPSCs, are engineered to express or overexpress SPI1. In some embodiments, pluripotent stem cells, such as hPSCs or hiPSCs, are engineered to express or overexpress CEBPA. some embodiments, pluripotent stem cells, such as hPSCs or hiPSCs, are engineered to express or overexpress FLIL some embodiments, pluripotent stem cells, such as hPSCs or hiPSCs, are engineered to express or overexpress MEF2C. some embodiments, pluripotent stem cells, such as hPSCs or hiPSCs, are engineered to express or overexpress CEBPB. some embodiments, pluripotent stem cells, such as hPSCs or hiPSCs, are engineered to express or overexpress IRF8.

In some embodiments, pluripotent stem cells, such as hPSCs or hiPSCs, are engineered to express or overexpress SPI1, CEBPA, and FLI1. In some embodiments, pluripotent stem cells, such as hPSCs or hiPSCs, are engineered to express or overexpress SPI1, CEBPA, and FLI1 and one or more of MEF2C, CEBPB, and IRF8. In some embodiments, pluripotent stem cells, such as hPSCs or hiPSCs, are engineered to express or overexpress SPI1, CEBPA, and FLI1 and two or more of MEF2C, CEBPB, and IRF8. In some embodiments, pluripotent stem cells, such as hPSCs or hiPSCs, are engineered to express or overexpress SPI1, CEBPA, FLI1, MEF2C, CEBPB, and IRF8.

A cell “expressed” a particular protein if the level of the protein in the cell is detectable (e.g., using a known protein assay). A cell “overexpresses” a particular protein (e.g., engineered polynucleotide encoding the protein) if the level of the protein is higher than (e.g., at least 5%, at least 10%, or at least 20% higher than) the level of the protein expressed from an endogenous, naturally-occurring polynucleotide encoding the protein.

### Engineered Polynucleotides and Polypeptides

The pluripotent stem cells of the present disclosure, in some embodiments, comprise engineered polynucleotides. An engineered polynucleotide is a nucleic acid (e.g., at least two nucleotides covalently linked together, and in some instances, containing phosphodiester bonds, referred to as a phosphodiester backbone) that does not occur in nature. Engineered polynucleotides include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered polynucleotide may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a polynucleotide is a complementary DNA (cDNA). cDNA is synthesized from a single-stranded RNA (e.g., messenger RNA (mRNA) or microRNA (miRNA)) template in a reaction catalyzed by reverse transcriptase.

Engineered polynucleotides of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered polynucleotides may be used in accordance with the present disclosure.

In some embodiments, an engineered polynucleotide comprises a promoter operably linked to an open reading frame. A promoter is a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked.

In some embodiments, a promoter is an inducible promoter. An inducible promoter may be regulated in vivo by a chemical agent, temperature, or light, for example. Inducible promoters enable, for example, temporal and/or spatial control of gene expression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells). In some embodiments, the inducible promoter is a tetracycline-inducible promoter. In some embodiments, the inducible promoter is a doxycycline-inducible promoter. In other embodiments, a promoter is a constitutive promoter (active in vivo, unregulated).

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

Vectors used for delivery of an engineered polynucleotide include minicircles, plasmids, bacterial artificial chromosomes (BACs), and yeast artificial chromosomes. Transposon-based systems, such as the piggyBac™ system (e.g., Chen et al. Nature Communications. 2020; 11(1): 3446), is also contemplated herein.

A pluripotent stem cells, in some embodiments, comprises an engineered polynucleotide comprising an open reading frame encoding a protein selected from SPI1, CEBPA, FLI1, MEF2C, CEBPB, and IRF8. In some embodiments, the engineered polynucleotide comprises an open reading frame encoding SPI1. In some embodiments, the engineered polynucleotide comprises an open reading frame encoding CEBPA. In some embodiments, the engineered polynucleotide comprises an open reading frame encoding FLI1. In some embodiments, the engineered polynucleotide comprises an open reading frame encoding MEF2C. In some embodiments, the engineered polynucleotide comprises an open reading frame encoding CEBPB. In some embodiments, the engineered polynucleotide comprises an open reading frame encoding IRF8.

An engineered polynucleotide encoding comprising an open reading frame encoding SPI1 (also known as Transcription factor PU.1) (e.g., UniprotKB Accession No. P17947), in some embodiments, encodes a protein comprising the sequence of:

(SEQ ID NO: 1)
MLQACKMEGF PLVPPPSEDL VPYDIDLYQR
QTHEYYPYLS SDGESHSDHY WDFHPHHVHS
EFESFAENNF TELQSVQPPQ LQQLYRHMEL
EQMHVLDTPM VPPHPSLGHQ VSYLPRMCLQ
YPSLSPAQPS SDEEEGERQS PPLEVSDGEA
DGLEPGPGLL PGETGSKKKI RLYQFLLDLL
RSGDMKDSIW WVDKDKGTFQ FSSKHKEALA
HRWGIQKGNR KKMTYQKMAR ALRNYGKTGE
VKKVKKKLTY QFSGEVLGRG GLAERRHPPH

An engineered polynucleotide encoding comprising an open reading frame encoding CCAAT/enhancer-binding protein alpha (CEBPA) (e.g., UniprotKB Accession No. P49715), in some embodiments encodes a protein comprising the sequence of:

(SEQ ID NO: 2)
MESADFYEAE PRPPMSSHLQ SPPHAPSSAA
FGFPRGAGPA QPPAPPAAPE PLGGICEHET
SIDISAYIDP AAFNDEFLAD LFQHSRQQEK
AKAAVGPIGG GGGGDFDYPG APAGPGGAVM
PGGAHGPPPG YGCAAAGYLD GRLEPLYERV
GAPALRPLVI KQEPREEDEA KQLALAGLFP
YQPPPPPPPS HPHPHPPPAH LAAPHLQFQI
AHCGQTTMHL QPGHPTPPPT PVPSPHPAPA
LGAAGLPGPG SALKGLGAAH PDLRASGGSG
AGKAKKSVDK NSNEYRVRRE RNNIAVRKSR
DKAKQRNVET QQKVLELTSD NDRLRKRVEQ
LSRELDTLRG IFRQLPESSL VKAMGNCA

An engineered polynucleotide encoding comprising an open reading frame encoding Friend leukemia integration 1 transcription factor (FLI1) (e.g., UniprotKB Accession No. Q01543), in some embodiments, encodes a protein comprising the sequence of:

(SEQ ID NO: 3)
MDGTIKEALS VVSDDQSLED SAYGAAAHLP
KADMTASGSP DYGQPHKINP LPPQQEWINQ
PVRVNVKREY DHMNGSRESP VDCSVSKCSK
LVGGGESNPM NYNSYMDEKN GPPPPNMTIN
ERRVIVPADP TLWTQEHVRQ WLEWAIKEYS
LMEIDTSFFQ NMDGKELCKM NKEDFLRATT
LYNTEVLLSH LSYLRESSLL AYNTTSHTDQ
SSRLSVKEDP SYDSVRRGAW GNNMNSGLNK
SPPLGGAQTI SKNTEQRPQP DPYQILGPTS
SRLANPGSGQ IQLWQFLLEL LSDSANASCI
TWEGINGEFK MTDPDEVARR WGERKSKPNM
NYDKLSRALR YYYDKNIMTK VHGKRYAYKF
DFHGIAQALQ PHPTESSMYK YPSDISYMPS
YHAHQQKVNF VPPHPSSMPV TSSSFFGAAS
QYWTSPTGGI YPNPNVPRHP NTHVPSHLGS
YY

An engineered polynucleotide encoding comprising an open reading frame encoding Myocyte-specific enhancer factor 2C (MEF2C) (e.g., UniprotKB Accession No. Q06413), in some embodiments, encodes a protein comprising the sequence of:

(SEQ ID NO: 4)
MGRKKIQITR IMDERNRQVT FTKRKFGLMK
KAYELSVLCD CEIALIIFNS INKLFQYAST
DMDKVLLKYT EYNEPHESRT NSDIVETLRK
KGLNGCDSPD PDADDSVGHS PESEDKYRKI
NEDIDLMISR QRLCAVPPPN FEMPVSIPVS
SHNSLVYSNP VSSLGNPNLL PLAHPSLQRN
SMSPGVTHRP PSAGNTGGLM GGDLTSGAGT
SAGNGYGNPR NSPGLLVSPG NLNKNMQAKS
PPPMNLGMNN RKPDLRVLIP PGSKNTMPSV
SEDVDLLLNQ RINNSQSAQS LATPVVSVAT
PTLPGQGMGG YPSAISTTYG TEYSLSSADL
SSLSGENTAS ALHLGSVTGW QQQHLHNMPP
SALSQLGACT STHLSQSSNL SLPSTQSLNI
KSEPVSPPRD RTTTPSRYPQ HTRHEAGRSP
VDSLSSCSSS YDGSDREDHR NEFHSPIGLT
RPSPDERESP SVKRMRLSEG WAT

An engineered polynucleotide encoding comprising an open reading frame encoding CCAAT/enhancer-binding protein beta (CEBPB) (e.g., UniprotKB Accession No. P17676), in some embodiments, encodes a protein comprising the sequence of:

(SEQ ID NO: 5)
MQRLVAWDPA CLPLPPPPPA FKSMEVANFY
YEADCLAAAY GGKAAPAAPP AARPGPRPPA
GELGSIGDHE RAIDESPYLE PLGAPQAPAP
ATATDTFEAA PPAPAPAPAS SGQHHDELSD
LESDDYGGKN CKKPAEYGYV SLGRLGAAKG
ALHPGCFAPL HPPPPPPPPP AELKAEPGFE
PADCKRKEEA GAPGGGAGMA AGFPYALRAY
LGYQAVPSGS SGSLSTSSSS SPPGTPSPAD
AKAPPTACYA GAAPAPSQVK SKAKKTVDKH
SDEYKIRRER NNIAVRKSRD KAKMRNLETQ
HKVLELTAEN ERLQKKVEQL SRELSTLRNL
FKQLPEPLLA SSGHC

An engineered polynucleotide encoding comprising an open reading frame encoding Interferon regulatory factor 8 (IRF8) (e.g., UniprotKB Accession No. Q02556), in some embodiments, encodes a protein comprising the sequence of:

(SEQ ID NO: 6)
MCDRNGGRRL RQWLIEQIDS SMYPGLIWEN
EEKSMFRIPW KHAGKQDYNQ EVDASIFKAW
AVFKGKFKEG DKAEPATWKT RLRCALNKSP
DFEEVTDRSQ LDISEPYKVY RIVPEEEQKC
KLGVATAGCV NEVTEMECGR SEIDELIKEP
SVDDYMGMIK RSPSPPEACR SQLLPDWWAQ
QPSTGVPLVT GYTTYDAHHS AFSQMVISFY
YGGKLVGQAT TTCPEGCRLS LSQPGLPGTK
LYGPEGLELV RFPPADAIPS ERQRQVTRKL
FGHLERGVLL HSSRQGVFVK RLCQGRVFCS
GNAVVCKGRP NKLERDEVVQ VEDTSQFFRE
LQQFYNSQGR LPDGRVVLCF GEEFPDMAPL
RSKLILVQIE QLYVRQLAEE AGKSCGAGSV
MQAPEEPPPD QVFRMFPDIC ASHQRSFFRE
NQQITV

The number of copies of an engineered polynucleotide delivered to a PSC may vary. In some embodiments, a PSC comprises 1-20 copies of an engineered polynucleotide. For example, and PSC may comprise 1-15, 1-10, 2-10, 2-15, 2-10, 5-20, 5-15, or 5-10 copies of an engineered polynucleotide. In some embodiments, a PSC comprises 8-10 copies of an engineered polynucleotide. Greater than 20 copies are also contemplated herein.

In some embodiments, an engineered polynucleotide encodes variant of a SPI1, CEBPA, FLI1, MEF2C, CEBPB, or IRF8 protein. Protein variants are proteins (including full length proteins and peptides) that differ in their amino acid sequence relative to a wild-type, native, or reference amino acid sequence. A protein variant may possess one or more substitutions, deletions, and/or insertions at certain positions within its amino acid sequence, as compared to a wild-type, native, or reference amino acid sequence. Ordinarily, protein variants have at least 50% identity to a wild-type, native or reference sequence. In some embodiments, a protein variant has at least 60%, at least 70%, at least 75%, at least 80%, at least 85%, at least 90%, or at least 95% identity to a wild-type, native, or reference sequence.

A protein variant may contain amino acid changes that confer any of a number of desirable properties, for example, that enhance its expression and/or improve its stability. Protein variants can be made using routine mutagenesis techniques and assayed as appropriate to determine whether they possess the desired property. Assays to determine expression levels of proteins, including protein variants, are well known in the art. The stability of a protein variant may be measured by assaying thermal stability or stability upon urea denaturation or may be measured using in silico prediction, for example. Methods for such experiments and in silico determinations are known in the art.

In some embodiments, an engineered polynucleotide comprises an open reading frame that encodes a protein comprising an amino acid sequence of any one of the sequences provided herein or comprises an amino acid sequence that has at least 50%, at least 60%, at least 70%, at least 75%, at least 80%, at least 85%, at least 90%, or at least 95% identity to the amino acid sequence of any one of the sequences provided herein. See, e.g., SEQ ID NOs: 1-6.

“Identity” refers to a relationship between two or among three or more sequences (e.g., amino acid sequences or nucleotide sequences) as determined by comparing the sequences to each other. Identity also refers to the degree of sequence relatedness between or among sequences as determined by the number of matches between or among strings of amino acids (polypeptides) or strings of nucleotides (polynucleotides). Identity is a measure of the percent of identical matches between the smaller of two or more sequences with gap alignments (if any) addressed by a particular mathematical model or computer program (e.g., “algorithms”). Identity of related polypeptides and polynucleotides can be readily calculated by known methods. “Percent (%) identity” as it applies to polypeptide or polynucleotide sequences is defined as the percentage of residues (amino acid or nucleic acid residues) in the candidate (first) polypeptide or polynucleotide sequence that are identical with the residues in a second polypeptide or polynucleotide sequence after aligning the sequences and introducing gaps, if necessary, to achieve the maximum percent identity.

Methods and computer programs for the alignment are well known in the art. It is understood that identity depends on a calculation of percent identity but may differ in value due to gaps and penalties introduced in the calculation. Generally, variants of a particular polynucleotide or polypeptide have at least 90%, 91%, 92%, 93%, 94%, 95%, 96%, 97%, 98%, 99% but less than 100% sequence identity to that particular wild-type, native, or reference sequence as determined by sequence alignment programs and parameters described herein and known to those skilled in the art. Such tools for alignment include but are not limited to those of the BLAST suite (Altschul, S. F., et al. Nucleic Acids Res. 1997; 25:3389-3402); and those based on the Smith-Waterman algorithm (Smith, T. F. & Waterman, M. S. J. Mol. Biol. 1981; 147:195-197). A general global alignment technique based on dynamic programming is the Needleman-Wunsch algorithm (Needleman, S. B. & Wunsch, C. D. J. Mol. Biol. 1920; 48:443-453). A Fast Optimal Global Sequence Alignment Algorithm (FOGSAA) also has been developed that purportedly produces global alignment of nucleotide and protein sequences faster than other optimal global alignment methods, including the Needleman-Wunsch algorithm.

As such, polynucleotides and polypeptides containing substitutions, insertions and/or deletions (e.g., indels), and covalent modifications with respect to wild-type, native, or reference sequence, for example, the polypeptide (e.g., protein) sequences disclosed herein, are included within the scope of this disclosure. For example, sequence tags or amino acids, such as one or more lysine(s), can be added to polypeptide sequences (e.g., at the N-terminal and/or C-terminal end). Sequence tags can be used for peptide detection, purification and/or localization. Lysines can be used to increase peptide solubility or to allow for biotinylation. Alternatively, amino acid residues located at the N-terminal and/or C-terminal regions of the amino acid sequence of a protein may optionally be deleted providing for truncated sequences.

As recognized by those skilled in the art, protein fragments, functional protein domains, and homologous proteins are also considered to be within the scope of proteins provided herein. For example, provided herein is any protein fragment of (meaning a polypeptide sequence at least one amino acid residue shorter than but otherwise identical to) a wild-type, native, or reference sequence, provided that the fragment retains its wild-type function. In addition to protein variants that are identical to the wild-type, native, or reference protein but are truncated, in some embodiments, a protein includes 2, 3, 4, 5, 6, 7, 8, 9, 10, or more mutations (e.g., substitutions, insertions and/or deletions), as shown in any of the sequences provided or referenced herein. Protein variants can range in length from about 4, 6, or 8 amino acids to full length proteins.

### Polycistronic Polynucleotides

In some embodiments, a pluripotent stem cell comprises one or more polycistronic polynucleotides. A polycistronic polynucleotide encodes several proteins on a single polynucleotide molecule. In some embodiments, a pluripotent stem cell comprises an engineered polycistronic polynucleotide comprising an open reading frame encoding SPI1, an open reading frame encoding CEBPA, and an open reading frame encoding FLI1. In some embodiments, a pluripotent stem cell comprises an engineered polycistronic polynucleotide comprising an open reading frame encoding MEF2C, an open reading frame encoding CEBPB, and an open reading frame encoding IRF8.

The order in which an open reading frame is encoded on a polycistronic polynucleotide can impact the expression level of the protein. Typically, the open reading frame placed at the upstream (5′) position, relative to the other open reading frames, is expressed at the highest levels. In some embodiments, a polycistronic polynucleotide comprises, 5′ to 3′ (in the following order), an open reading frame encoding SPI1, an open reading frame encoding CEBPA, and an open reading frame encoding FLI1. In other embodiments, a polycistronic polynucleotide comprises, 5′ to 3′ (in the following order), an open reading frame encoding SPI1, an open reading frame encoding FLI1, and an open reading frame encoding CEBPA. In some embodiments, a polycistronic polynucleotide comprises, 5′ to 3′ (in the following order), an open reading frame encoding MEF2C, an open reading frame encoding CEBPB, and an open reading frame encoding IRF8. In other embodiments, a polycistronic polynucleotide comprises, 5′ to 3′ (in the following order), an open reading frame encoding MEF2C, an open reading frame encoding IRF8, and an open reading frame encoding CEBPB.

In some embodiments, the open reading frames are separated from each by a sequence encoding a self-cleaving peptide, such as a 2A self-cleaving peptide (e.g., T2A, P2A, E2A, and F2A). In some embodiments, the open reading frames are separated from each by an internal ribosome entry site (IRES).

### Methods of Producing Microglia-Like Cells

The methods of producing microglia-like cells provided herein, in some aspects, comprises culturing, in culture media, a population of pluripotent stem cells (PSCs) to produce an expanded population of PSCs; and expressing in PSCs of the expanded population a protein selected from SPI1, CEBPA, FLI1, MEF2C, CEBPB, and IRF8 (e.g., SPI1, CEBPA, and FLI1; or SPI1, CEBPA, FLI1, MEF2C, CEBPB, and IRF8) to produce microglia-like cells.

In some embodiments, the PSCs of the expanded population comprise an engineered polynucleotide comprising an open reading frame encoding SPI1. In some embodiments, the PSCs of the expanded population comprise an engineered polynucleotide comprising an open reading frame encoding CEBPA. In some embodiments, the PSCs of the expanded population comprise an engineered polynucleotide comprising an open reading frame encoding FLI1. In some embodiments, the PSCs of the expanded population comprise an engineered polynucleotide comprising an open reading frame encoding MEF2C. In some embodiments, the PSCs of the expanded population comprise an engineered polynucleotide comprising an open reading frame encoding CEBPB. In some embodiments, the PSCs of the expanded population comprise an engineered polynucleotide comprising an open reading frame encoding IRF8.

In some embodiments, the PSCs of the expanded population comprise an engineered polycistronic polynucleotide comprising an open reading frame encoding SPI1, an open reading frame encoding CEBPA, and an open reading frame encoding FLI1. In some embodiments, the PSCs of the expanded population further comprise an engineered polycistronic polynucleotide comprising an open reading frame encoding MEF2C, an open reading frame encoding CEBPB, and an open reading frame encoding IRF8.

In some embodiments, an open reading frame of the engineered polynucleotide is operably linked to a heterologous promoter.

In some embodiments, the heterologous promoter is an inducible promoter, non-limiting examples of which are provided elsewhere herein.

The population a starting population comprises about 1×10²-1×10¹⁰, about 1×10²-1×10⁹, about 1×10²-1×10⁸, or about 1×10²-1×10⁷ PSCs. In some embodiments, the population comprises about 1×10³-1×10⁸ or about 1×10³-1×10⁷ PSCs. In some embodiments, the population comprises about 1×10⁴-1×10⁷ or about 1×10⁵-1×10⁶ PSCs. In some embodiments, the population comprises about 1×10¹ PSCs, about 1×10² PSCs, about 1×10³ PSCs, about 1×10⁴PSCs, about 1×10⁵PSCs, about 1×10⁶PSCs, about 1×10⁷PSCs, about 1×10⁸ PSCs, about 1×10⁹ PSCs, or about 1×10¹⁰ PSCs.

In some embodiments, the population of PSCs or an expanded population of PSCs is cultured for about 4 to about 10 days, about 4 to about 9 days, about 4 to about 8 days, about 4 to about 7 days, about 4 to about 6 days, about 5 to about 10 days, about 5 to about 9 days, about 5 to about 8 days, about 5 to about 7 days, or about 5 to about 6 days. In some embodiments, the population of PSCs is cultured for about 4 days, about 5 days, about 6 days, about 7 days, about 8 days, about 9 days, or about 10 days.

Some methods of the present disclosure provide methods comprising (a) delivering to PSCs one or more engineered polynucleotides comprising an inducible promoter operably linked to one or more open reading frame encoding a protein selected from SPI1, CEBPA, FLI1, MEF2C, CEBPB, and IRF8; (b) culturing the PSCs in feeder-free, serum-free culture media to produce an expanded population of PSCs; and (c) culturing PSCs of the expanded population in an induction media comprising an inducing agent to produce CD11b⁺, CX3CR1⁺, ITGAM⁺, P2RY12⁺, TMEM119⁺, TREM2⁺, TRA-1-60⁻ and/or POU5F1⁻ microglia-like cells.

In some embodiments, the PSCs are cultured in feeder-free, serum-free culture media for about 6 to about 24 hours. For example, the PSC may be cultured in feeder-free, serum-free culture media for about, 6 to about 12 hours. In some embodiments, the PSCs are cultured in feeder-free, serum-free culture media for about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, the expanded population of PSCs comprises at least 5×10³ PSCs. For example, the expanded population (e.g., at the time of induction) may comprise at least 1×10⁴, at least 1×10⁵, at least 1×10⁶, or at least 1×10⁷ PSCs. In some embodiments, the expanded population of PSCs comprises about 5×10³ PSCs to about 1×10⁷ PSCs.

In some embodiments, PSCs of the expanded population are cultured at a density of about 10,000 cells/cm² to about 30,000 cells/cm². In some embodiments, PSCs of the expanded population are cultured at a density of about 10,000 cells/cm² to about 25,000 cells/cm². In some embodiments, PSCs of the expanded population are cultured at a density of about 10,000 cells/cm² to about 20,000 cells/cm². In some embodiments, PSCs of the expanded population are cultured at a density of about 10,000 cells/cm² to about 15,000 cells/cm². In some embodiments, PSCs of the expanded population are cultured at a density of about 15,000 cells/cm² to about 30,000 cells/cm². In some embodiments, PSCs of the expanded population are cultured at a density of about 15,000 cells/cm² to about 25,000 cells/cm². In some embodiments, PSCs of the expanded population are cultured at a density of about 15,000 cells/cm² to about 20,000 cells/cm². In some embodiments, PSCs of the expanded population are cultured at a density of at least 10,000/cm², at least 15,000/cm², at least 20,000/cm², at least 25,000/cm², or at least 30,000/cm².

In some embodiments, PSCs of the expanded population are cultured for no longer than 10 days, no longer than 9 days, no longer than 8 days, no longer than 7 days, no longer than 6 days, no longer than 5 days, or no longer than 4 days. For example, PSCs of the expanded population may be cultured for about 4 to about 10 days, about 4 to about 9 days, about 4 to about 8 days, about 4 to about 7 days, about 4 to about 6 days, about 5 to about 10 days, about 5 to about 9 days, about 5 to about 8 days, about 5 to about 7 days, or about 5 to about 6 days. In some embodiments, PSCs of the expanded population are cultured for about 4 days, about 5 days, about 6 days, about 7 days, about 8 days, about 9 days, or about 10 days.

In some embodiments, PSCs of the expanded population are cultured in an induction media for about 36 to about 60 hours. For example, the PSC may be cultured in an induction media for about 36 to about 54 hours, about 36 to about 48 hours, about 42 to about 60 hours, about 42 to about 54 hours, about 42 to about 48 hours, about 48 to about 60 hours, or about 48 to about 54 hours. In some embodiments, the PSCs are cultured in an induction media for about 36 hours, about 42 hours, about 48 hours, about 54 hours, or about 60 hours.

Culturing in the second induction media comprises, in some embodiments, several (one or more) media changes. For example, an induction media may be removed and replaced with new (fresh) second indication media every (about) 12 hours, every 24, hours, every 36 hours, or every 48 hours. In some embodiments, an induction media is changed every (about) 24 hours.

### Transfection Methods

The engineered polynucleotide of the present disclosure may be delivered to a PSC using any one or more transfection method, including chemical transfection methods, viral transduction methods, and electroporation.

In some embodiments, an engineered polynucleotide is delivered on a vector. A vector is any vehicle, for example, a virus or a plasmid, that is used to transfer a desired polynucleotide into a host cell, such as a PSC. In some embodiments, the vector is a viral vector. In some embodiments, a viral vector is not a naturally occurring viral vector. The viral vector may be from adeno-associated virus (AAV), adenovirus, herpes simplex virus, lentiviral, retrovirus, varicella, variola virus, hepatitis B, cytomegalovirus, JC polyomavirus, BK polyomavirus, monkeypox virus, Herpes Zoster, Epstein-Barr virus, human herpes virus 7, Kaposi's sarcoma-associated herpesvirus, or human parvovirus B 19. Other viral vectors are encompassed by the present disclosure.

In some embodiments, a viral vector is an AAV vector. AAV is a small, non-enveloped virus that packages a single-stranded linear DNA genome that is approximately 5 kb long and has been adapted for use as a gene transfer vehicle (Samulski, R J et al., Annu Rev Virol. 2014; 1(1):427-51). The coding regions of AAV are flanked by inverted terminal repeats (ITRs), which act as the origins for DNA replication and serve as the primary packaging signal (McLaughlin, S K et al. Virol. 1988; 62(6): 1963-73; Hauswirth, W W et al. 1977; 78(2):488-99). Thus, an AAV vector typically includes ITR sequences. Both positive and negative strands are packaged into virions equally well and capable of infection (Zhong, L et al. Mol Ther. 2008; 16(2):290-5; Zhou, X et al. Mol Ther. 2008; 16(3):494-9; Samulski, R J et al. Virol. 1987; 61(10):3096-101). In addition, a small deletion in one of the two ITRs allows packaging of self-complementary vectors, in which the genome self-anneals after viral uncoating. This results in more efficient transduction of cells but reduces the coding capacity by half (McCarty, D M et al. Mol Ther. 2008; 16(10): 1648-56; McCarty, D M et al. Gene Ther. 2001; 8(16): 1248-54).

In some embodiments, a polynucleotide is delivered to a cell using a transposon/transposase system. For example, the piggyBac™ transposon system may be used. A piggyBac™ transposon is a mobile genetic element that efficiently transposes between vectors and chromosomes via a “cut and paste” mechanism (Woodard et al. 2015). During transposition, the piggyBac™ transposase recognizes transposon-specific inverted terminal repeat sequences (ITRs) located on both ends of the transposon vector and efficiently moves the contents from the original sites and integrates them into TTAA chromosomal sites. The piggyBac™ transposon system facilitates efficient integration of a polynucleotide into a cell genome.

Thus, in some embodiments, the method further comprises delivering to a PSC a transposon comprising an engineered polynucleotide and also delivering a transposase.

In some embodiments, an engineered polynucleotide is delivered to a cell using electroporation. Electroporation is a physical transfection method that uses an electrical pulse to create temporary pores in cell membranes through which the engineered polynucleotide can pass into cells. See, e.g., Chicaybam L et al. Front. Bioeng. Biotechnol., 23 Jan. 2017.

Following transfection, the engineered polynucleotides may be integrated into the genome of a PSC. In some embodiments, an engineered polynucleotide may further comprise an antibiotic resistance gene to confer resistance to an antibiotic used in an antibiotic drug selection process. In this way, a ‘pure’ population of cells comprising an integrated engineered polynucleotide may be obtained. In some embodiments, a population of cells comprising an integrated engineered polynucleotide are selected using antibiotic drug selection. Antibiotic drug selection is the process of treating a population of cells with an antibiotic so that only cells that are capable of surviving in the presence of said antibiotic will remain in the population. Non-limiting examples of antibiotics that may be used for antibiotic drug selection include: puromycin, blasticidin, geneticin, hygromycin, mycophenolic acid, zeocin, carbenicillin, kanamycin, ampicillin, and actinomycin.

### Culture Media

The methods provided herein, in some embodiments, comprise culturing PSCs in a feeder-free, serum-free culture media. Culture media may comprise, for example, a solubilized basement membrane preparation extracted from the Engelbreth-Holm-Swarm (EHS) mouse sarcoma (e.g., Corning® Matrigel® Matrix) (coated at ˜75 μl/cm² to ˜150 μl/cm² of lot-based diluted suspension). In some embodiments, the solubilized basement membrane preparation comprises one or more extracellular matrix (ECM) protein and one or more growth factor. For example, the ECM proteins may be selected from Laminin, Collagen IV, heparan sulfate proteoglycans, and entactin/nidogen.

In some embodiments, the culture media further comprises one or more growth factor, for example, selected from recombinant human basic fibroblast growth factor (rh bFGF) (e.g., 80 ng/ml to 120 ng/ml) and recombinant human transforming growth factor β (rh TGFβ) (e.g., 20 pM to 25 pM). In some embodiments, culture media further comprises rh bFGF and rh TGFβ. In some embodiments, culture media comprises mTeSR™ Plus medium (STEMCELL Technologies).

In some embodiments, the culture media further comprises a small molecule ROCK inhibitor (e.g., 9 μM to 11 μM), such as Y-27632, to facilitate seeding the of PSCs.

In some embodiments, an inducing agent is added to the culture media to produce an induction media. In some embodiments, the induction media comprises an inducing agent (e.g., doxycycline (e.g., 0.1-1 μg/mL, optionally about 0.5 μg/ml)).

### Therapeutic Compositions and Method of Use

The present disclosure provides, in some embodiments, therapeutic compositions comprising the microglia-like cells produced herein. In some embodiments, the compositions further comprise a pharmaceutically-acceptable excipient. The compositions, in some embodiments, are cryopreserved.

Such compositions may be administered to a subject, such as a human subject, using any suitable route of administration. Suitable routes of administration include, for example, parenteral routes such as intravenous, intrathecal, parenchymal, or intraventricular routes. Suitable routes of administration include, for example, parenteral routes such as intravenous, intrathecal, parenchymal, or intraventricular injection.

In some embodiments, a subject is a human subject.

Patient with spinal cord injury (SCI) may benefit from the use of these cells. After differentiation of microglia-like cells from iPSCs, the cells can be stimulated with IL-4 to acquire a pro-tissue repair and anti-inflammatory phenotype. The simulated microglia-like cells can then be transplanted to sites near the injury and promote neuronal repair and improve motor function.

iPSC-derived microglia can also be used as disease models for studying disease pathology and screening for therapeutic molecules. For example, microglia can be added to a neuronal cell culture for studying the effects of immune cells on neuronal networks under disease stimulations (i.e., beta amyloid, Tau protein, TDP43). Microglia can be added to three dimensional cultures like organoids to provide a more faithful model for brain development and degeneration. To model neurological diseases with possible genetic background, i.e. Alzheimer's disease, amyotrophic lateral sclerosis, or multiple sclerosis, microglia-like cells can be differentiated from patient-derived iPSCs to accurately reflect patients' genetic background and allow genetic stratification during drug screening.

The compositions may be administered to a subject in a therapeutically effective amount. The term “therapeutically effective amount” refers to the number of granulosa required to confer therapeutic effect on a subject, either alone or in combination with at least one other active agent. Effective amounts vary, as recognized by those skilled in the art, depending on the route of administration, excipient usage, and co-usage with other active agents. The quantity to be administered depends on the subject to be treated, including, for example, the strength of an individual's immune system or genetic predispositions. Suitable dosage ranges are readily determinable by one skilled in the art and may be on the order of micrograms of the polypeptide of this disclosure. The dosage of the preparations disclosed herein may depend on the route of administration and varies according to the size of the subject.

It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited in the present application are incorporated by reference for the purposes or subject matter referenced in this disclosure.

### EXAMPLES

Recent advances and applications of single-cell assays, exemplified by collaborative efforts such as the Human Cell Atlas (HCA), have begun to provide a comprehensive view of cell types and cellular states within the human body. Such maps are crucial for understanding human development and diseases. From a synthetic biology perspective, these maps can be mined for promising targets for cell fate engineering, with significant implications for disease modeling, cell therapy, and regenerative medicine. Previously, an unbiased approach was used for screening a comprehensive human transcription factor library (TFome), and 290 transcription factors (TFs) that induced differentiation of human induced pluripotent stem cells (hiPSCs) into various cell types were identified. While the unbiased screening method led to many interesting discoveries, it does not guarantee the generation of any particular cell type. For those wishing to differentiate stem cells into a specific cell type of interest for studying diseases and creating therapeutics, the availability of experimental and computational pipelines for the identification of TFs to produce target cell types would be of great benefit. In the Examples below, a target cell type for which TF-based differentiation method has not yet been found, the microglia, was selected for developing new screening methodologies.

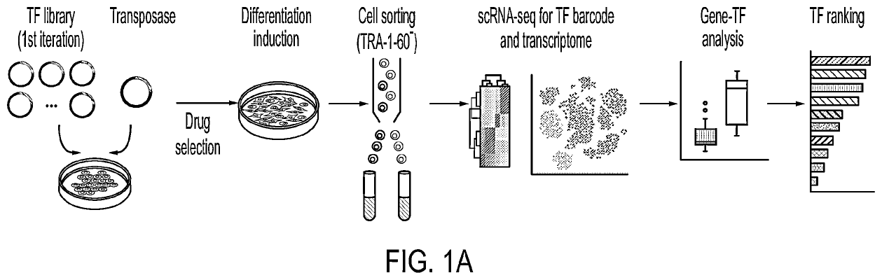

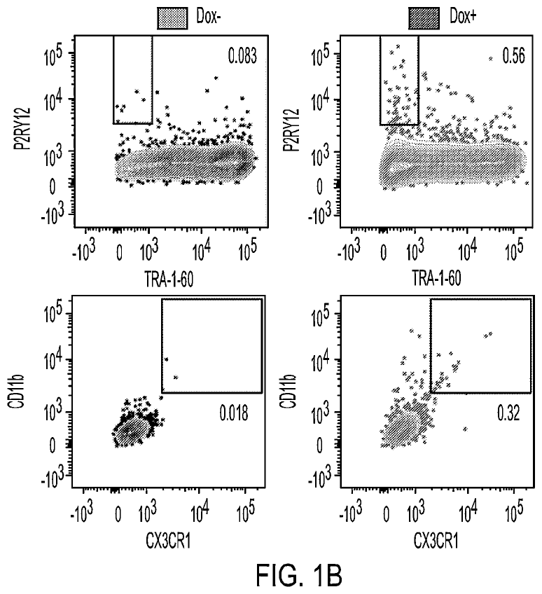

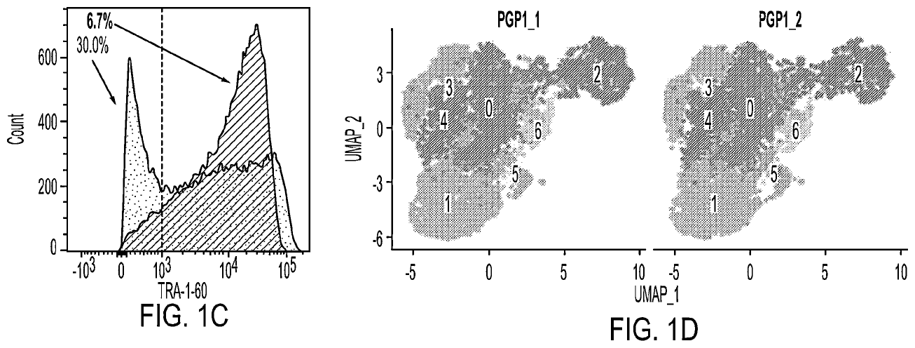

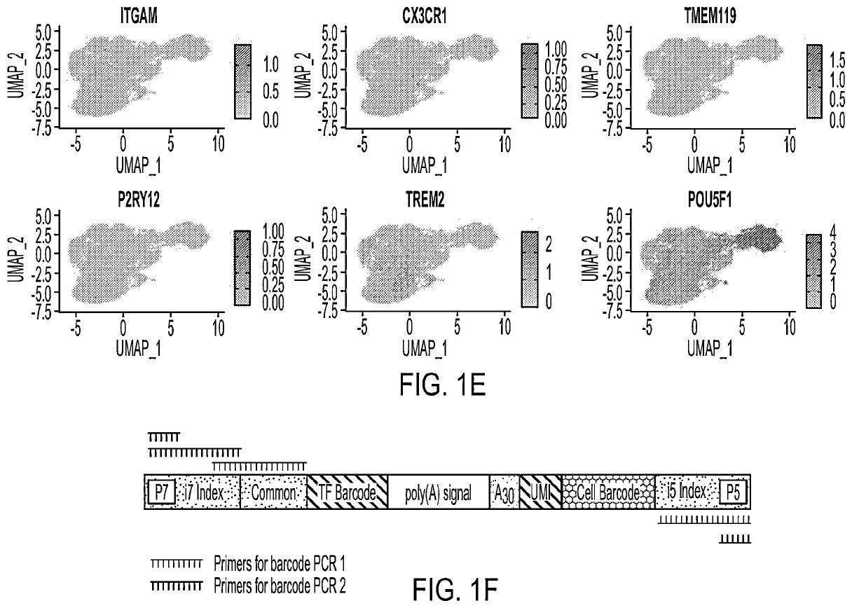

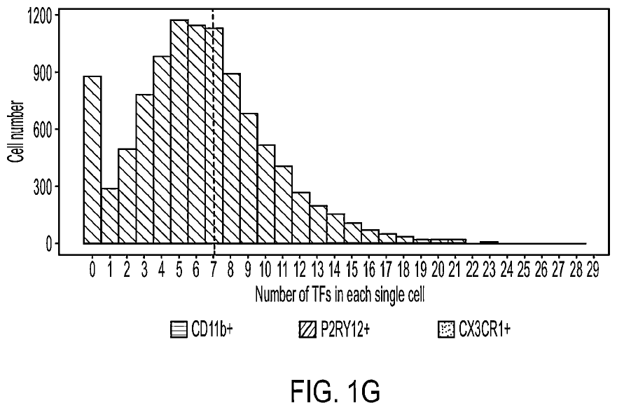

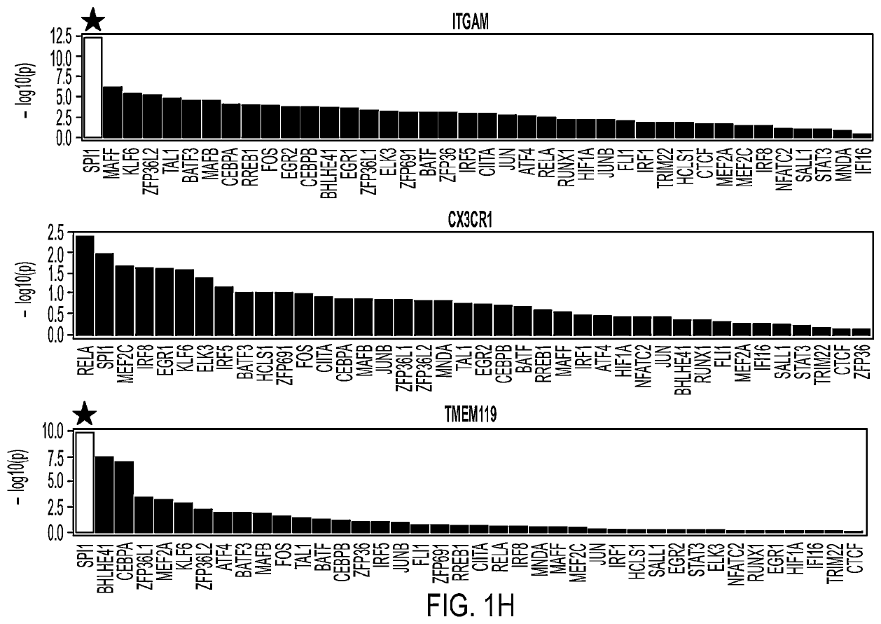

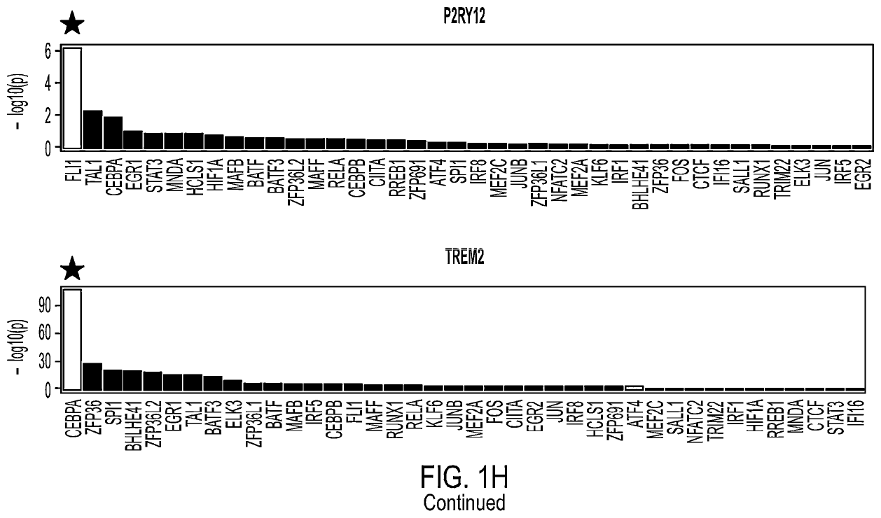

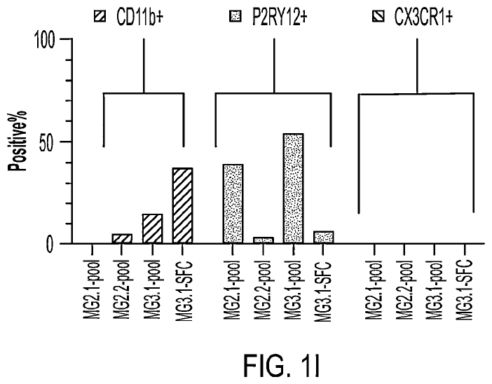

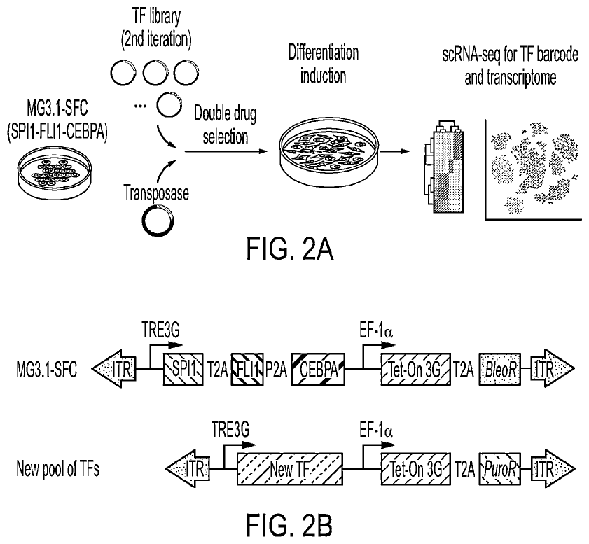

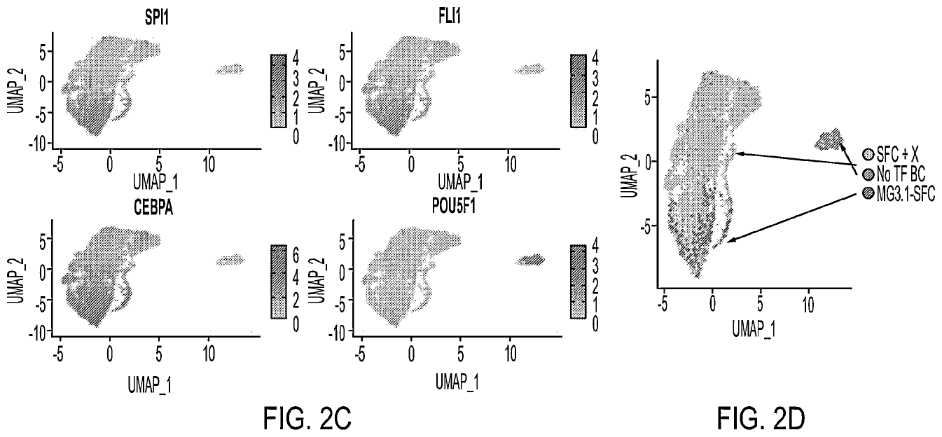

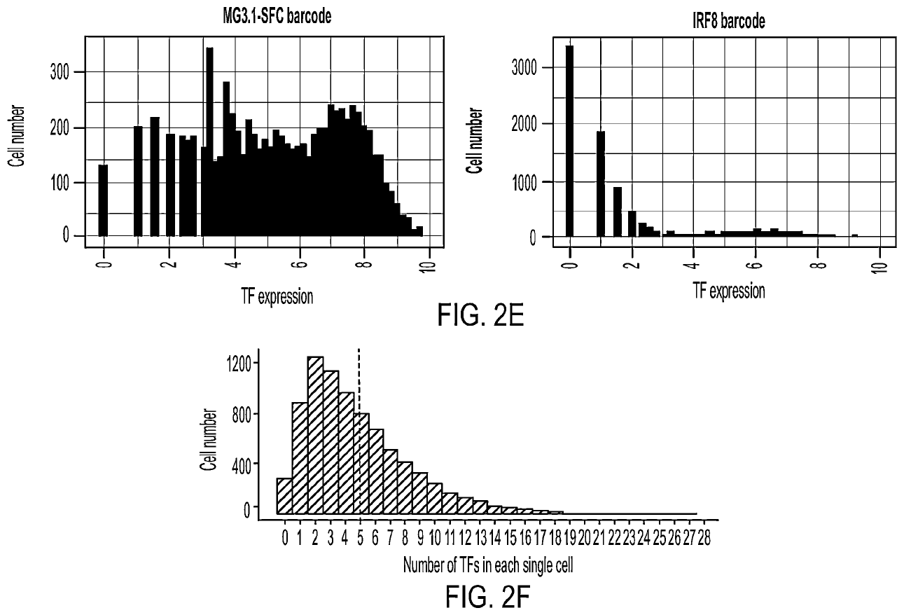

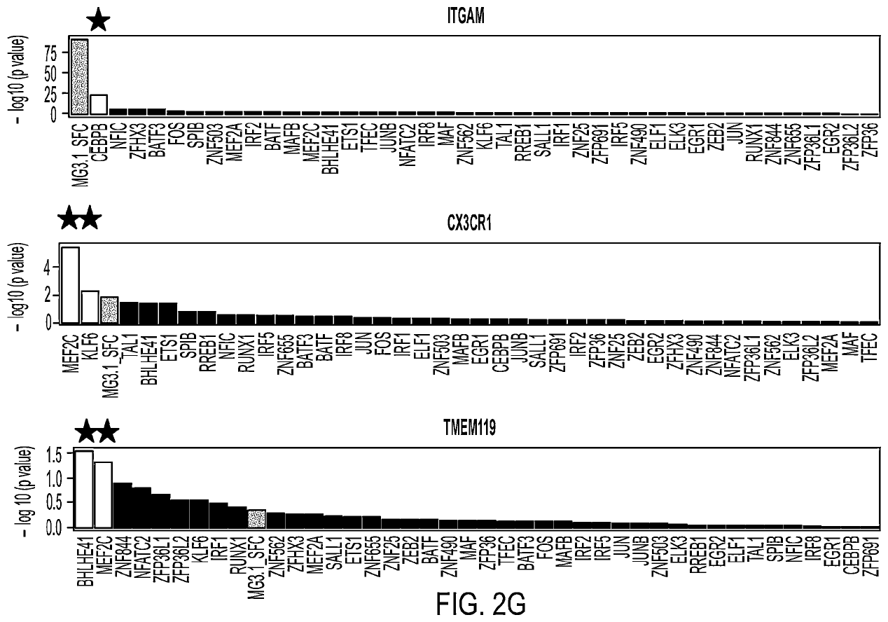

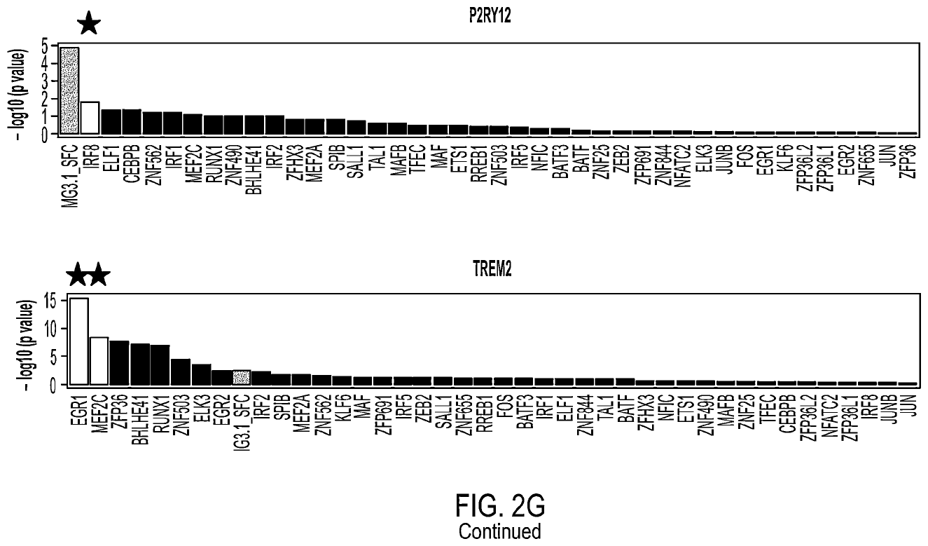

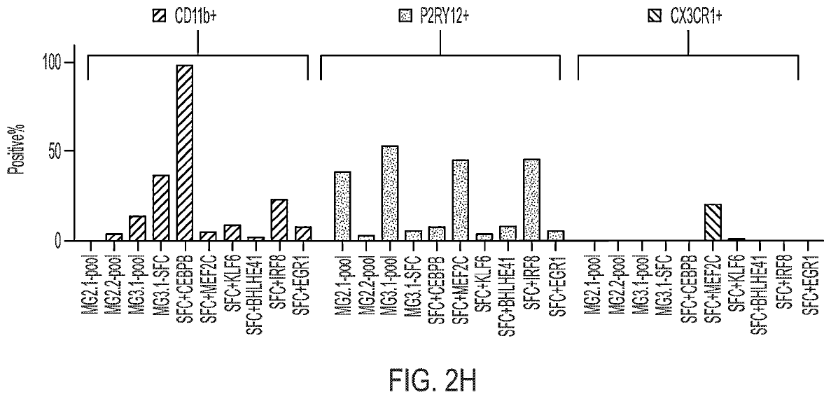

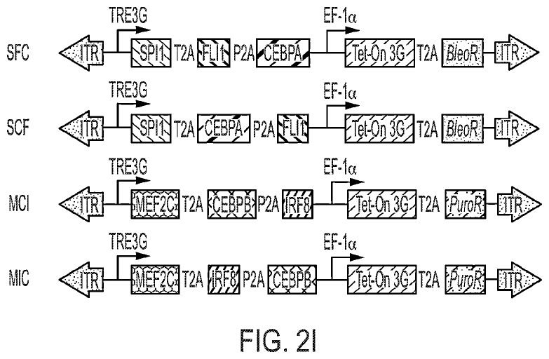

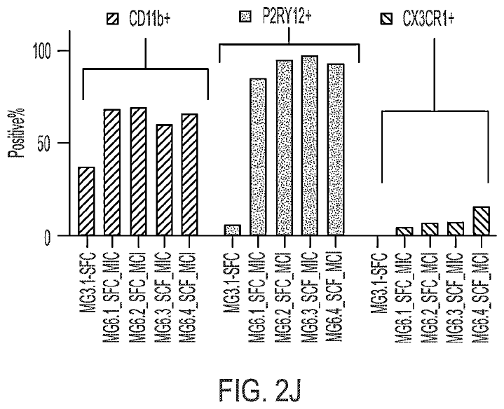

In the Examples below, two sequential iterations of pooled TF screening were conducted. Each round of screening involved creating a barcoded TF library, pooled transfection into iPSCs for inducing differentiation, and subsequent single-cell transcriptome analysis. From the analysis, TFs were ranked by their ability to induce microglial gene expression, and the top hits were then characterized for their ability to induce differentiation into microglia (FIG. 1A, FIG. 2A). The analysis identified a TF combination that produced, in four days without the need for media exchange, cells that transcriptionally resembled microglial cells. These TF-induced microglia-like cells (TFiMGLs) shared molecular and functional features of human primary microglia. The barcoding and amplification strategy allows for simultaneous detection of cell and TF barcodes from the high throughput single-cell experiments, thus empowering an analysis of the regulatory relationships between TFs and other genes. A human single-cell transcriptome reference was also constructed by integrating publicly available scRNA-seq datasets of 225 samples representing 59 tissue types, to which TF differentiated single cells could be mapped. The methodology described in this study should be broadly adopted to cell fate engineering and enable researchers to more effectively select TFs to generate novel iPSC-derived cell types.

### Example 1. The First Pooled Screening Identified Initial TFs for Inducing Microglia Gene Expression

To identify TFs that differentiate hiPSCs to microglia, the overall strategy is to first transfect and integrate stochastically a pool of TFs into the cells, followed by differentiation induction and single-cell RNA sequencing (scRNA-seq). From the scRNA-seq data, TFs enriched in cells differentiating towards microglia, based on gene expression, were identified. Forty (40) candidate TFs were cloned into the pBAN2 vector for genomic integration with PiggyBac transposase and doxycycline (Dox)-inducible expression. To distinguish between exogenous and endogenous transcripts, a 20-nucleotide (nt) barcode was added between the stop codon and the poly-A sequence of each TF. The 40 TF vectors were transfected into 600,000 hiPSCs from a healthy donor (PGP1) with a 4:1 mass ratio of TF to transposase DNA, which lead cells to uptake and integrate multiple TFs. After puromycin selection for TF-integrated cells, differentiation was induced by the additional of Dox for four days (FIG. 1A). Flow cytometry analysis showed that 0.3-0.5% of the cells expressed microglial surface proteins, including CX3CR1, P2RY12, and CD11b (FIG. 1B).

To pinpoint which of the 40 TF(s) were inducing microglial gene expression, all differentiated cells for scRNA-seq were sorted (FIG. 1A). After four days of differentiation, 30% of the cells lost expression of a stem cell marker, TRA-1-60 (FIG. 1C). Two independent transfections were performed (data not shown), and each group was spiked in with around 10% non-induced cells as undifferentiated control during scRNA-seq. Expression of microglia genes (ITGAM, P2RY12, CX3CR1, TMEM119, TREM2) was observed, as well as a cluster of cells with high expression of POU5F1, marking stem cells (FIGS. 1D, 1E). Through amplicon sequencing of co-amplified TF and cell barcodes from cDNAs (FIG. 1F), it was possible to quantify the amount of exogeneous TF(s) expressed in single cells (data not shown). An average of seven (7) TFs was expressed per cell, and 877 (8.5%) out of 10285 single cells had no TF expression, which agreed with the 10% stem cell spike-in (FIG. 1G). By comparing TF expression levels in cells with or without microglial RNA expression, it was possible to identify which TF(s) have a higher expression in cells with microglial RNA. Three (3) TFs were identified as likely to cause microglial gene expression, including SPI1, FLI1, and CEBPA (FIG. 1H). SPI1, which encodes PU.1 protein, is a known TF required for microglia development. CEBPA is a known critical regulator for myeloid differentiation. And FLI1, while less reported for microglia development, has been reported to interact with RUNX140 and SPI141, where both TFs are indispensable for tissue-resident macrophage development.

Individually expression of CEBPA and FLI1 in hiPSCs led to almost complete cell death (data not shown), indicating their expression level needs to be tightly controlled. SPI1 alone only induced around 3% cells positive for CD11b (data not shown), indicating additional TFs are needed for the differentiation. Pooled transfection of CEBPA+FLI1 (MG2.1-pool) or CEBPA+SPI1 (MG2.2-pool) led to improved microglial marker expression, while CEBPA+FLI1+SPI1 (MG3.1-pool) produced the most positive cells, reaching 14% CD11b+, 54% P2RY12+ after four days (FIG. 1I). However, no expression of CX3CR1, a chemokine receptor important for microglia activation and migration (43,44), was observed. Because pooled transfection and PiggyBac integration of three plasmids does not guarantee each cell to have all three TFs, polycistronic expression cassettes were constructed by linking the TFs with 2A self-cleaving peptides. Gene position in the cassette affects their relative expression level, with the first gene being the highest (Liu, Z. et al. Sci. Rep. 7, 2193 (2017)). The TFs were arranged in different orders and the constructs named based on the TF initials' order. For example, SPI1-T2A-FLI1-P2A-CEBPA was named “MG3.1-SFC”. Among MG3.1-CFS, —FCS and —SFC, the first two constructs led to dramatic cell death by day 4 (data not shown), an observation that is consistent with the fact that CEBPA and FLI1 caused cell death by themselves. MG3.1-SFC, which positioned SPI1 at the front, produced 37% CD11b+ and 6% P2RY12+ cells, while still no expression of CX3CR1 (FIG. 1I). The differences between MG3.1-SFC and MG3.1-pool is potentially due to different dosages of the TFs. While MG3.1-pool allows for variable dosage combination of the three TFs among different cells, MG3.1-SFC produces a fixed dosage for all cells. Irrespective of the dosage variation, the lack of CX3CR1 expression and the overall low percentage of CD11b and P2RY12 positive cells indicate that additional TFs are needed for driving microglia differentiation from hiPSCs.

### Example 2. The Second Iteration Identified Additional TFs for Microglia Differentiation

To build upon the hits from the first pooled screen and to identify more TFs that can promote microglia differentiation, a second pooled screen was performed on top of the three TFs from the first iteration (3+X) (FIG. 2A). The second TF pool contained a total of 42 candidate TFs. To ensure each cell received both SPI1-T2A-FLI1-P2A-CEBPA and additional TFs from the second pool, bleomycin was used in addition to puromycin for selection of both constructs (FIG. 2B). Similar to the first pooled screen, 600,000 PGP1 hiPSCs were transfected and drug-selected in duplicates. After four (4) days of Dox-induced differentiation, the cell pool was subjected to the same single-cell RNA and TF barcode amplicon sequencing as in the first iteration, except no cell sorting was used. As controls, 5% undifferentiated hiPSCs and 5% MG3.1-SFC were spiked in during single-cell encapsulation to mark the differentiation starting point of two iterations. TF barcode analysis did reveal two clusters of cells on UMAP that corresponded to hiPSCs and MG3.1-SFC, while also showed new clusters of cells that express additional TFs (FIG. 2C, 2D), indicating altered differentiation with new TFs. Based on TF barcode counting, out of 8051 single cells from two independent transfections, 549 (6.8%) cells had no TF barcode and 613 (7.6%) cells had only the barcode for MG3.1-SFC. On average each cell expressed five TFs (FIG. 2E, 2F), with most of the cells expressing the SFC cassette plus at least one other TF.

To determine if any of the new TFs are improving microglia differentiation, their effects on microglial RNA expression we analyzed, especially CX3CR1, which was not expressed in MG3.1-SFC. A significantly higher number of MEF2C and KLF6 barcode in cells expressing CX3CR1 (FIG. 2G) was observed, suggesting their ability to induce CX3CR1 expression. MEF2C was also present in the first screening but failed to reach significance for upregulating CX3CR1, indicating the presence of the SFC triple TF cassette was helpful. MEF2C also reached high ranking for TMEM119 (FIG. 2G) but was lower than BHLHE41. The SFC cassette ranked highest for inducing ITGAM and P2RY12 expression, which is expected from the results of the first iteration. CEBPB and IRF8 also demonstrated potential to promote ITGAM or P2RY12 expression (FIG. 2G). In summary, additional TFs of interest include MEF2C, CEBPB, IRF8, KLF6, and BHLHE41.

To validate the additional TFs can promote microglial gene expression, they were individually expressed with SFC (SFC+1). When compared with MG3.1-SFC, CEBPB increased the percentage of CD11b+ cells from 37% to 98% (FIG. 2H) but led to more cell death at day 4. MEF2C and IRF8 increased P2RY12 expression from 6% to around 45% (FIG. 2H). Most importantly, MEF2C and KLF6 increased CX3CR1+ cells from none to 20% and 2% (FIG. 2H). These results agreed well with the predictions from single-cell TF barcode analysis, indicating the validity of using pooled TF screening for inferring causality between TF and target gene expression. To test whether microglia differentiation can be further promoted by delivering more TFs to each cell, we combined MEF2C, CEBPB and IRF8 into polycistronic cassettes. Because MEF2C demonstrated ability to induce both CX3CR1 and P2RY12, it was placed first and the position of CEBPB and IRF8 was varied, producing two cassettes: MIC and MCI (FIG. 2I). The position of FLI1 and CEBPA was also varied in the first construct to produce SFC and SCF, keeping SPI1 in the front to avoid excessive cell death during differentiation. All four combinations for the tri-TF cassettes (SFC-MIC, SFC-MCI, SCF-MIC, SCF-MCI) were tested for their ability to induce microglia differentiation (FIG. 2I). Encouragingly, all hexa-TF cocktails produced cell pools with increased expression of microglial proteins when compared with MG3.1-SFC (FIG. 2J). The best cocktail is MG6.4-SCF-MCI, containing 66% CD11b+, 93% P2RY12+ and 16% CX3CR1+ cells at day 4, while for MG3.1-SFC these number were 37%/6%/0%. These results highlight the significance of the second iteration and demonstrated the utility of iterative TF screening for cell fate engineering.

### Example 3. TFiMGLs Differentiate Quickly, are Phagocytic, Responsive to Disease-Relevant Stimulation, and Share Molecular Signatures with Primary Microglia

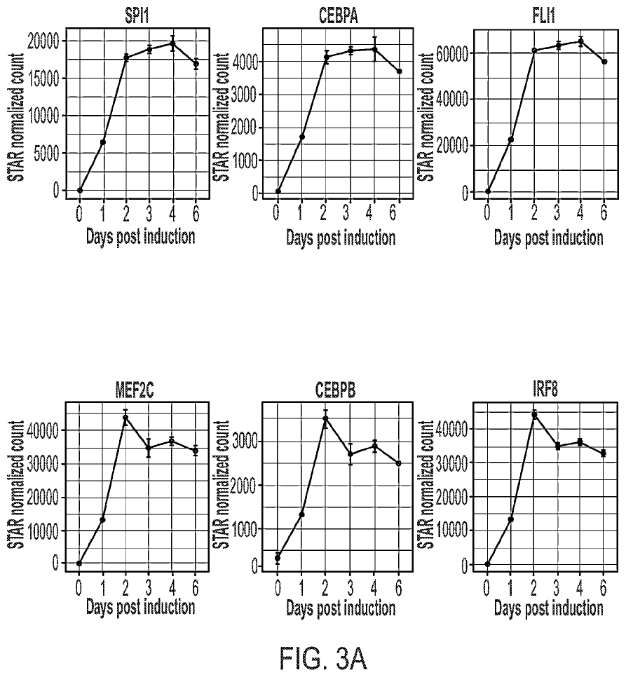

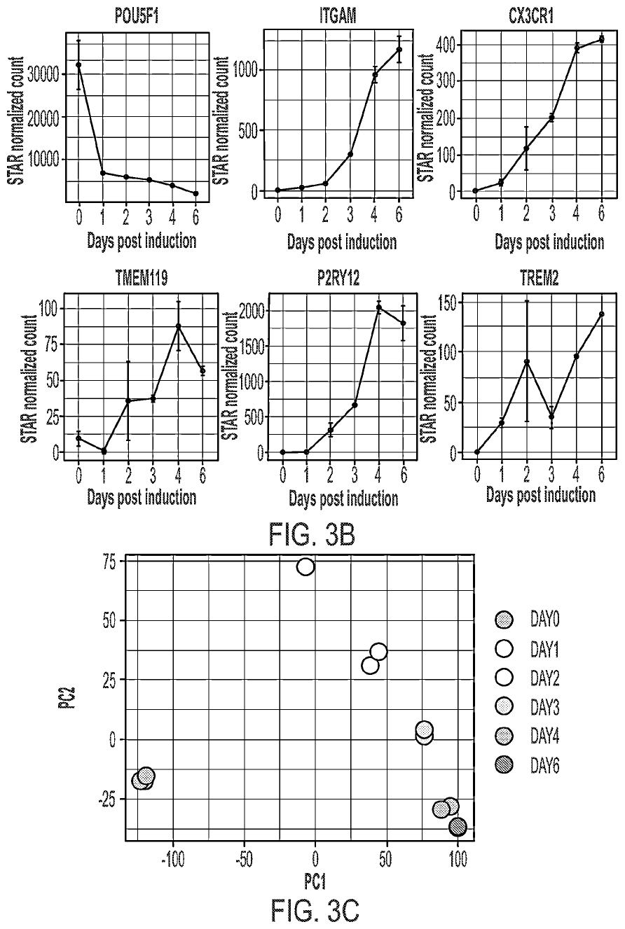

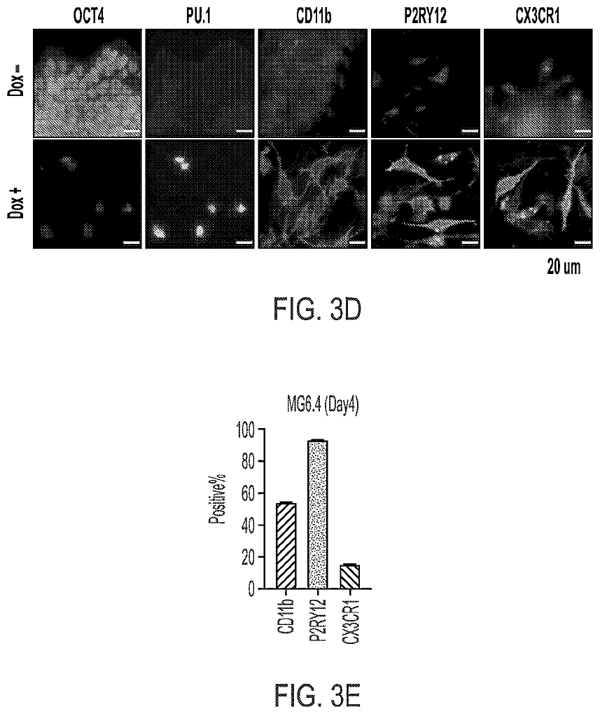

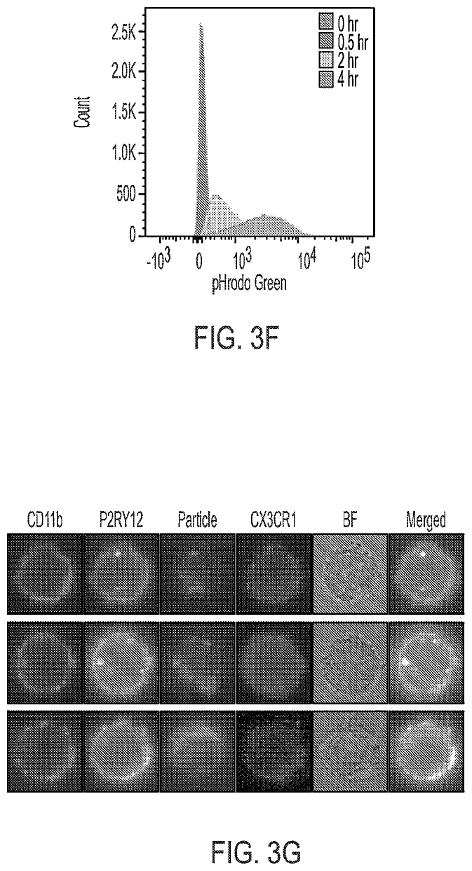

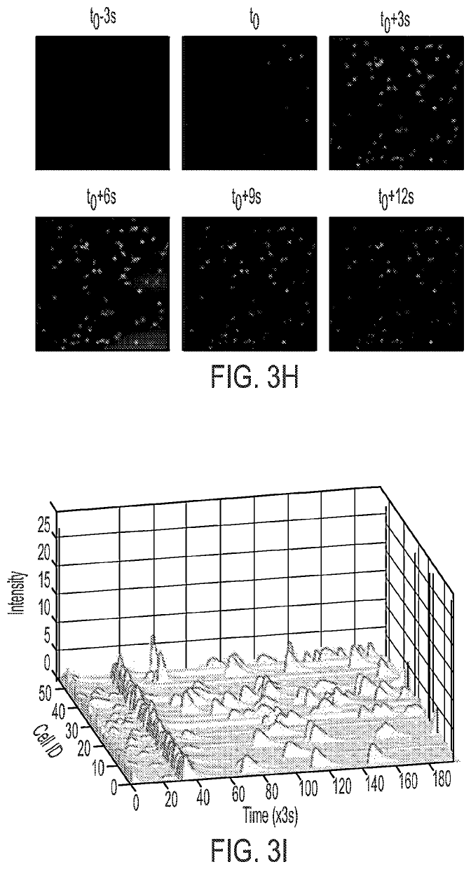

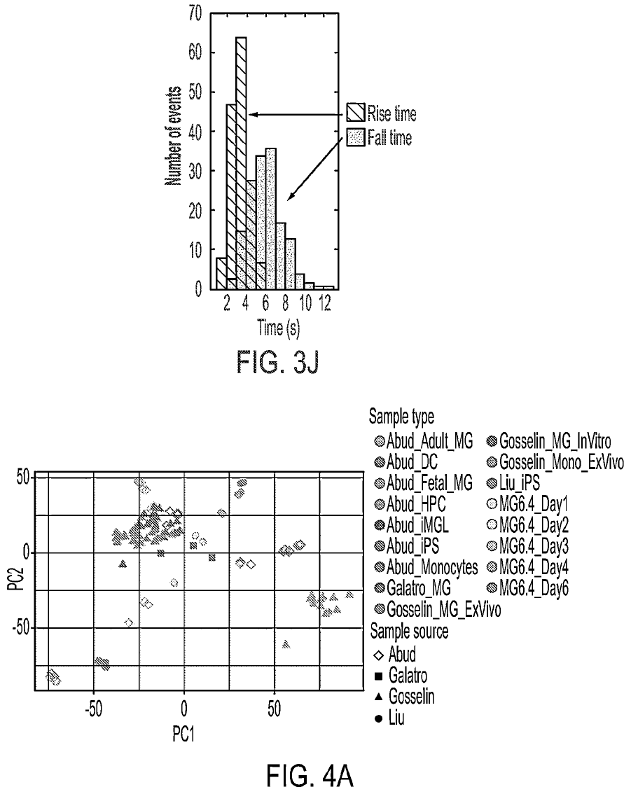

To determine the differentiation dynamics of MG6.4, bulk RNA-seq analysis of the cells was performed on 0, 1, 2, 3, 4, 6 days post induction. A rapid induction of the six TFs was observed on day 1, reaching a plateau on day 2 (FIG. 3A). This was accompanied by a quick downregulation of POU5F1 on day 1, followed by upregulation of microglial genes from day 2 onwards (FIG. 3B). Principal component analysis (PCA) reflected a similar trend, where a rapid differentiation occurred on day 1 and 2, followed by a gradual deceleration from day 3 to day 6 (FIG. 3C). The transcriptome on day 4 and 6 reside closely together, indicating a stable window for functional studies and applications. Thus, for downstream characterizations of MG6.4, all differentiation was carried out for 4 days.

Brightfield microscopy analysis of MG6.4 revealed rapid morphological change from day 1 to day 6 (data not shown). Immunofluorescence analysis confirmed the expression of key TFs and microglial proteins (FIG. 3D). MG6.4 demonstrated reproducible differentiation between replicates, with 53.9±0.57% CD11b+, 93.1±0.50% P2RY12+ and 14.8±0.68% CX3CR1+ cells (FIG. 3E). As brain resident macrophages, microglia play important roles in brain development and homeostasis. Microglia's abilities to respond to signals related to degenerating neurons and phagocytose are integral parts of their function. To investigate if MG6.4 could mimic the phagocytosis function of microglia, MG6.4 was incubated with pHrodo green labeled S. aureus particles for 0.5, 2 and 4 hours, followed by flow cytometry and microscopy analysis. While 0.5-hour incubation showed minimal phagocytosis activity, nearly all cells were positive for pHrodo green at 2 hours and the intensity grew even stronger at 4 hours (FIG. 3F). Microscopy analysis at 4 hours with co-staining of microglia surface proteins confirmed the intracellular position of these particles (FIG. 3G). To study if MG6.4 is responsive to ADP stimulation, day 4 MG6.4 was pre-incubated with calcium indicator Fluo-4 and stimulated with ADP containing media while being imaged every three second. A rapid increase in calcium signal was observed when ADP was added (FIG. 3H, 3I, 3J), suggesting MG6.4 is responsive to ADP stimulation.

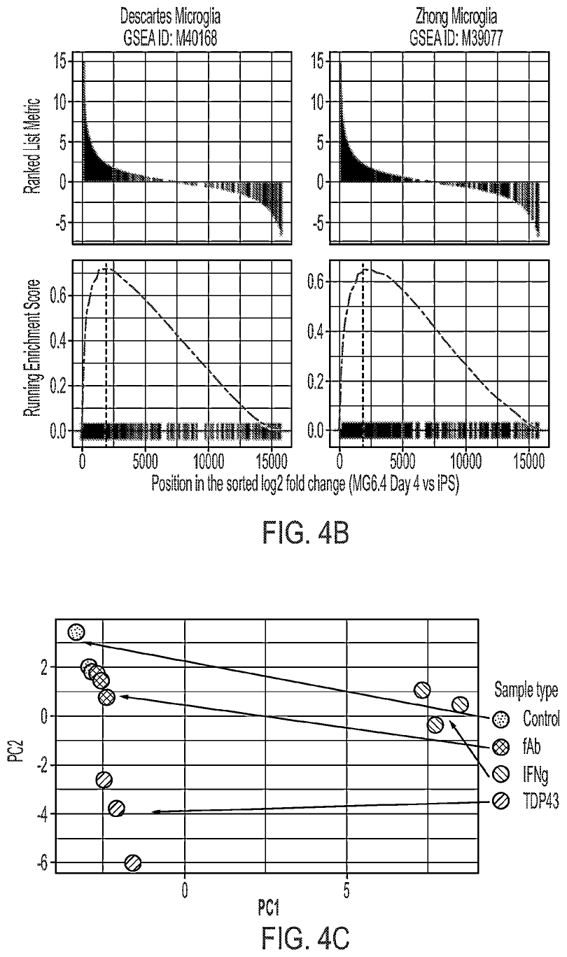

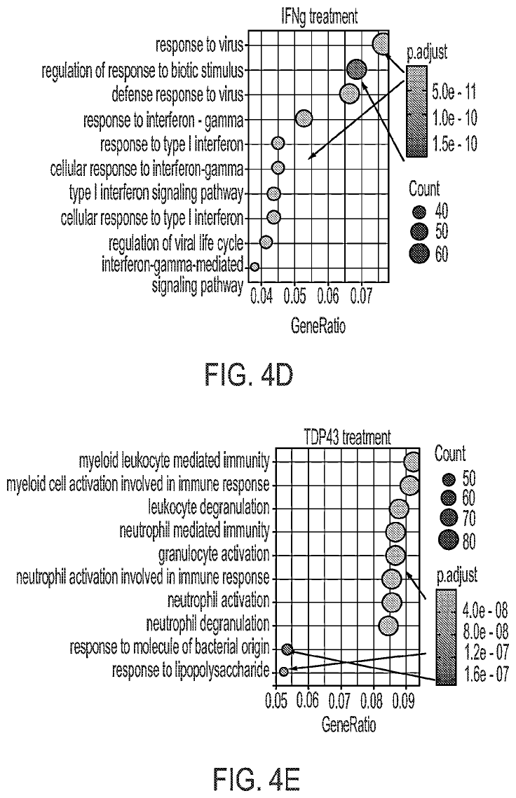

To assess how TFiMGLs recapitulate the transcriptome of human microglia, previously published bulk RNA-seq data for human primary microglia and iPSC-derived microglia were downloaded (Abud, E. M. et al. Neuron 94, 278-293.e9 (2017); Gosselin, D. et al. Science 356, (2017); Galatro, T. F. et al. Nat. Neurosci. 20, 1162-1171 (2017)). To address potential batch problems that may hinder meaningful comparison between datasets, all raw FASTQ files were aligned to the same reference genome and a negative binomial regression-based batch effect correction method, ComBat-seq50, was applied before downstream analysis. Based on the PCA analysis, MG6.4 on day 2-6 resided closer to primary microglia of different sources than to iPSCs or monocytes, indicating a successful microglial fate induction (FIG. 4A). scRNA-seq data from primary human brain enabled the definition of microglial-enriched gene sets. Using two microglial gene collections within the MSigDB derived from human brain scRNA-seq, Gene Set Enrichment Analysis (GSEA) (Subramanian, A. et al. Proc. Natl. Acad. Sci. USA 102, 15545-15550 (2005)) was performed on day 4 MG6.4 versus iPSCs. Positive microglial gene enrichment scores were observed using both collections, indicating MG6.4 upregulates those microglia-enriched genes when compared to iPSCs (FIG. 4B). A collection of 881 microglia-enriched genes (Gosselin, D. et al. Science 356, (2017)) was used to cluster the samples from FIG. 4A. While day-1 MG6.4 still clustered closer to iPSCs, day-2 and later MG6.4 clustered closer to primary microglia, with most genes upregulated to a greater extent on day 4 and 6 than day 2 and 3 (data not shown). These results indicate that MG6.4 was able to establish a microglia-like transcriptome starting from day 2.

Microglia are able to respond to signals indicating brain infection and inflammation. IFNγ is a known activator of microglia secreted by T lymphocyte. Beta amyloid (Aβ) is a key molecule in AD pathology shown to elicit microglia response. TDP-43, the aggregation of which is considered a hallmark of ALS and is present in the vast majority of ALS patient, was also shown to activate microglia. To investigate how TFiMGLs respond to IFNγ, fibrillar A3 (fAβ) and TDP-43, day 4 MG6.4 was treated in triplicates with the stimulants for 24 hours and harvested for RNA-seq. PCA analysis revealed transcriptomic changes in the IFNγ and TDP-43 treated group, while the fAβ-treated group showed minimal differences (FIG. 4C). fAβ formation was confirmed by in vitro amyloid fibrillation experiment that showed the AR peptide could form fibrils after 1 hour of incubation (data not shown). Pathway analysis of differentially expressed genes from the IFNγ treated group included “response to virus” and “response to bacterium” (FIG. 4D), which is expected because IFNγ is produced by T cell in response to infection. Top upregulated genes by IFNγ included CXCL10, CXCL11, IRF1 and IL18BP (data not shown). For the TDP-43 treated cells, top differentially regulated pathway included “myeloid leukocyte mediated immunity” and “myeloid cell activation involved in immune response” (FIG. 4E), further confirming the activation of MG6.4 by TDP-43. Top upregulated genes by TDP-43 included CXCL1, SOD2, NFKBIZ, CCL20 and C3 (data not shown). Collectively, these results suggest that MG6.4 exhibited expected response to infection and ALS-related stimulations. The lack of response to fAβ treatment remains to be investigated.

### Example 4. Single-Cell Atlas Reference Mapping Identified Causal TFs for Multiple Cell Fates

Using a group of cellular markers to determine cell type is a common practice in both primary human tissue and stem cell differentiation studies. Recent progresses in single-cell analysis technologies made it possible to define cell types based on more comprehensive molecular profiles, including transcriptome, epigenome, proteome and metabolome. To investigate how single-cell atlas data could be used to guide cell fate engineering efforts, there are several prerequisites that are currently only partially satisfied: 1) existence of single-cell data from all human tissue types, ideally also different developmental stages; 2) data integration methods to combine datasets from different sources for creating a comprehensive cell atlas; 3) reference mapping methods to project new dataset (e.g., iPSC-derived cells) onto the atlas and quantitatively score their similarity to all classes; 4) existence of perturbation libraries that are scRNA-seq compatible, which include but not limited to open reading frame (ORF) and CRISPR libraries.

To explore this idea of atlas-guided cell fate engineering using the previously described two pooled TF screen for microglia differentiation, scRNA-seq data from published datasets generated through 10× Chromium platform were compiled, with majority of the data from PanglaoDB55, where all raw reads from different studies were aligned and processed together. Two other organs that were under-represented in PanglaoDB, brain and endometrium, were added. In total, the final single-cell atlas contains 225 samples from 59 organ/tissue types, with a total of 1,004,650 single cells (data not shown). All raw data downloaded from PanglaoDB went through careful filtering in Seurat for cell, gene, UMI number and mitochondria gene ratio (data not shown). At its current status, this atlas is annotated to organ/tissue levels, while acquiring cellular level annotation from all 59 studies could be a work for future study to refine this atlas. To reduce batch variations resulted from different studies, the data were integrated with two different pipelines, Cluster Similarity Spectrum (CSS) or Harmony (data not shown). Qualitative assessment of the UMAP plots post integration does indicate co-clustering of cells in related tissues from different studies, exemplified by the mixing of “Primary brain” with “Embryo forebrain” datasets, and “Pancreatic islets” with “Pseudoislets” datasets (data not shown). Next, the scRNA-seq data from the two pooled TF screens was projected onto the integrated atlas using CSS or Symphony60 (data not shown). Nearest neighbor (NN) analysis in the CSS- or Harmony-corrected meta feature space revealed the mostly likely primary tissue type the TF-driven cells bear resemblance to (data not shown).

Being identified as NN or co-localized on UMAP plots does not, at least in the scenario of TF-driven iPSC differentiation described herein, mean that the engineered cells shared the exact transcriptome of primary cells. This interpretation is that when NNs are identified, the engineered cells on a relative scale with current sensitivity and accuracy, share the most gene expression features with their NNs when compared with all other reference cells. Even though during the first pooled screen cells with stem cell marker TRA-1-60+ were removed before scRNA-seq, the majority of the cells were mapped to the “iPSC/NPC” sample, indicating incomplete differentiation of those cells (data not shown). This proportion was consistently reduced in the second pooled screen when analyzed by computational pipelines (data not shown), which is expected because most cells are already integrated with the top three hits (SPI1, FLI1, CEBPA) from the first iteration. The driver TF(s) that cause engineered cells to be mapped on organ/tissue other than “iPSC/NPC” were then investigated. From both pipelines, a group of cells mapped to “Primary brain”. Considering microglia is the most abundant myeloid cells in the brain, it was hypothesized that these cells might be mapped to microglia. Acquiring cellular level annotation of the “Primary brain” dataset showed that these engineered cells reside closely to what's annotated as “microglia” from the study (data not shown). Analyzing the TF barcodes that were expressed higher in the “microglia” mapping group showed that CEBPA and CEBPB were significantly enriched (data not shown), partially confirming the hits identified from the two iterations of screen. CEBPA was also enriched in cells mapped to “Bone marrow whole”, “PBMC”, and “Monocyte-derived macrophages” samples (data not shown). CIITA was highly enriched in “Cord blood CD34” sample (data not shown). TFs enriched for other NNs were also analyzed (data not shown). Based on the exploratory experiment and analysis presented here, it is reasonable to expect with a larger perturbation library, a more refined single-cell atlas, and dedicated computational pipelines, it will be possible to explore cell fate engineering in more multiplexed ways.

### Methods

Barcoded TF expression vector construction. All TFs used in this study were obtained from the TFome collection in pDONR format. For expression in hiPSCs, a PiggyBac integrating Dox-inducible vector pBAN was used. To create barcoded pBAN expression vector (pBAN-BC), the original pBAN was digested with AgeI and KpnI, followed by ligation of a gBlock (IDT DNA) containing the same excised piece with an additional 20-bp random barcode. After bacteria transformation, individual colonies were expanded and extracted for plasmid DNA. Gateway cloning was used to transfer each TF from pDONR to pBAN-BC vector. Barcode sequence for each TF was confirmed by Sanger sequencing.
Cell culture. hiPSCs were culture in mTeSR Plus media (Stemcell Technologies, 100-0276) on multi-wells plates coated with Matrigel (Corning, 354277) or Cultrex (Bio-Techne Corporation, 3434-005-02). For passaging, cells were dissociated with TrypLE Express (Life Technologies, 12604013) and seeded into fresh plate and media containing 10 μM Y-27632 ROCK inhibitor (Millipore, 688001) for 24 hours. Daily media change was performed until cells were ready for another passaging or downstream experiments.
Nucleofection, TF integration and differentiation. TF (pBAN-TF-BC) and Super PiggyBac (SPB) Transposase (System Biosciences, PB210PA-1) expression vectors were mixed at a mass ratio of 4:1 and transfected into hiPSCs using P3 Primary Cell 4D-Nucleofector X Kit L (Lonza, V4XP-3024) on a 4D-Nucleofector X Unit (Lonza, AAF-1002X) following manufacturer's instructions. For the two pooled TF screenings, 600,000 cells were transfected with 5 μg of DNA and seeded into one well of a 6-well plate. For individual TF combinations, 120,000 cells were transfected with 2.5 μg of DNA and seeded into one well of a 12-well plate. Program CB150 was used for the nucleofections. 48 hours after nucleofection, 1 μg/mL of puromycin (Gibco, A1113803) or 50 μg/mL of zeocin (Gibco, R25001) was added to the culture for the selection of TF-integrated cells. Cells were passaged again when reaching 80% confluency. For induction of TF expression, cells were seeded into mTeSR Plus media containing 0.5 μg/mL doxycycline (Sigma-Aldrich, D3072) and 10 μM Y-27632 ROCK inhibitor and were changed into media only containing doxycycline after 24 hours.
Flow cytometry and sorting. For cytometry analysis, cells were dissociated with TrypLE Express for 5 minutes at 37 degree, diluted with twice the volume of Cell Staining Buffer (Biolegend, 420201) and centrifuged at 200 g for 3 minutes to remove the digesting enzyme. Cells were then incubated with 25 g/mL of Human Fc Block (BD Biosciences, 564219) diluted in Cell Staining Buffer for 15 minutes on ice, followed immediately by staining with fluorescently conjugated antibodies or isotype controls at proper dilution for 30 minutes on ice. Antibodies were diluted in Cell Staining Buffer and Human Fc Block was not removed from the mixture. After antibody staining, cells were washed twice with Cell Staining Buffer before being put through 35 μm nylon mesh into a 5 mL round bottom polystyrene tube (Falcon, 352235). Flow cytometry data was acquired on a BD LSRFortessa Cell Analyzer. For cell sorting, the staining protocol was the same except for that Cell Staining Buffer was replaced with mTeSR Plus media in order to maintain the best viability of cells. Cell sorting was performed on a BD FACSAria Cell Sorter. Flow cytometry antibodies used in this study were: FITC-TRA-1-60 (BD Biosciences, 560380), BV421-CX3CR1 (Biolegend, 341620), PE-P2RY12 (Biolegend, 392104), APC-CD11b (Biolegend, 101212). Isotype controls used were: BV421-Rat IgG2b (Biolegend, 400640), PE-Mouse IgG2a (Biolegend, 400214), APC-Rat IgG2b (Biolegend, 400612).
scRNA-seq library preparation. scRNA-seq experiments were performed using 10× Genomics Chromium Single Cell 3′ Reagent Kits v3 or v3.1 following the manufacturer's instruction. 5000 single cells were calculated as targeted input for each sample. For the first iteration, 10% of stem cells were spiked in as undifferentiated control. For the second iteration, 5% stem cells and 5% MG3.1-SFC were spiked in as undifferentiated and initial differentiation control. The only modification made to the protocol was at the Sample Index PCR step, where 5 μL of the PCR mix was taken out and mixed with 0.5 μL 1000×SYBR Gold (Invitrogen, S11494) for a qPCR reaction. The optimal amplification cycle was determined as the cycle just before half maximum of the total signal. Final libraries were sequenced on NextSeq 500 or NovaSeq with a goal of at least 30,000 reads per cell.
TF barcode amplicon library preparation. Because after the cDNA amplification step in the 10× scRNA-seq protocol the amplicons contained cell barcodes, UMIs, and TF barcodes, these cDNAs could be used as the template for further amplification of TF-cell barcodes. Two sequential PCR reactions were performed, each was accompanied by a SYBR Gold spike-in qPCR to determine the optimal cycle number as described in “scRNA-seq library preparation”. For PCR1, NGS10x-F-i7-BC-PCR1F and i5000 were used as primers. A 50 μL PCR1 reaction contains 25 μL Q5 Hot Start High-Fidelity 2× Master Mix (New England Biolabs, M0494L), 5 μL amplified cDNA, 2.5 μL of both primers at 10 μM stock concentration, and 15 μL nuclease-free water. PCR1 program was initial denaturation, 98 degrees, 30 seconds; 11-13 cycles (qPCR determined) of 98 degrees, 10 seconds, 67 degrees, 30 seconds, 72 degrees, 30 seconds; final extension, 72 degrees, 2 minutes. PCR1 reaction was purified with 1.2×SPRIselect beads (Beckman Coulter, B23318) following standard protocol. The sample was eluted in 20 μL water. For PCR2, i7000, P5, and P7 were used as primers. A 50 μL PCR2 reaction contains 25 μL Q5 Hot Start High-Fidelity 2× Master Mix, 10 μL PCR1 product, 2.5 μL of all three primers at 10 μM stock concentration, and 7.5 μL nuclease-free water. PCR2 program was initial denaturation, 98 degrees, 30 seconds; 4-5 cycles (qPCR determined) of 98 degrees, 10 seconds, 67 degrees, 30 seconds, 72 degrees, 30 seconds; final extension, 72 degrees, 2 minutes. PCR2 product was purified the same as PCR1. Final libraries were submitted for MiSeq v3 with paired-end reads of 80 cycles from either direction.

Primer sequences:
NGS10x-F-i7-BC-PCR1F:
(SEQ ID NO: 7)
GGAGTTCAGACGTGTGCTCTTCCGATCTCTTTT
CCAAGCACCTGCTACATAG
i5000:
(SEQ ID NO: 8)
AATGATACGGCGACCACCGAGATCTACACaact
cgctACACTCTTTCCCTACACGACGCTCTTCCG
ATCT
(lower case region represents
a sample-specific barcode)
i7000:
(SEQ ID NO: 9)
CAAGCAGAAGACGGCATACGAGATtcgccttaG
TGACTGGAGTTCAGACGTGTGCTCTTCCGATCT
(lower case region represents a
sample-specific barcode)
P5:
(SEQ ID NO: 10)
AATGATACGGCGACCACCGA
P7:
(SEQ ID NO: 11)
CAAGCAGAAGACGGCATACGA

Analysis of scRNA-seq and TF barcode-seq data. For scRNA-seq, raw FASTQ files were aligned to GRCh38 and quantified using Cell Ranger. Seurat was used to performed cell filtering, data normalization and clustering. The generated Seurat object also contained the single-cell raw expression matrix for all genes. For TF barcode-seq, in the paired-end MiSeq data, one of the read pair contains the 20 bp TF barcode while the other one contains the 16 bp cell barcode and the 12 bp UMI. By matching the names of the reads within the pair, three sequences were compiled into one table with three columns: TF-BC, cell-BC, UMI. To remove duplicated reads from the same molecule, duplicated rows that has the same value for all three columns were removed. Then the table was counted and reshaped into a frequency table where the row names represent cell and column names represent TF. This table contains the raw counts of each TF barcode in all single cells. Because the TF barcodes were amplified from the cDNA during library preparation, we normalized the TF barcode count with the number of total RNA UMIs detected in each cell, reasoning that cells with more total UMIs were likely to have more reads for TF barcode. The raw gene expression matrix and normalized TF count matrix were used to identify which TF barcodes were likely to induce microglial gene expression. Specifically, the expression of microglial genes was binarized, with any cell had a non-zero expression being 1. Then between the two groups of cells 0 or 1 microglial gene expression, a Wilcoxon rank sum test was performed for all barcoded TFs to determine which TF(s) had a higher expression in cells expressing microglial genes. The TFs were ranked by −log 10 (p-value).
Bulk RNA-seq library preparation. Cultured cells were dissolved directly with TRIzol (Thermo Fisher Scientific, 15596018) for total RNA purification with Direct-zol RNA MiniPrep Kit (Zymo Research, R2050). RNA concentration was quantified with Qubit RNA HS Assay Kit (Thermo Fisher Scientific, Q32852). RNA integrity was confirmed by presence of 18S and 28S bands on a 2% E-Gel EX Agarose Gel (Thermo Fisher Scientific, G402002). Between 100 ng to 1000 ng total RNA was used as input for mRNA enrichment using NEBNext Poly(A) mRNA Magnetic Isolation Module (New England Biolabs, E7490), followed by library construction with NEBNext Ultra II Directional RNA Library Prep Kit (New England Biolabs, E7760S) following the manufacturer's instructions. Biopolymers Facility at Harvard Medical School performed library QC and sequencing.
Analysis of bulk RNA-seq data. For both in-house generated sample and datasets downloaded from GEO, raw FASTQ files were aligned to GRCh38 and quantified using STAR 2.5.2b. Regularized-logarithm (rlog) transformation was applied to the raw counts before visualization using PCA. For analysis where data from multiple sources were involved, ComBat-seq was used for batch correction before PCA. Differential gene expression analysis was conducted with DESeq2⁴⁶. Pathway enrichment and GSEA analysis were performed with clusterProfiler⁷³.
Immunofluorescence (IF). IF experiments were performed in μ-Plate 96 Well Black plate (ibidi, 89626). After media removal, cells were fixed with 4% paraformaldehyde (Electron Microscopy Sciences, 15710) in 1× phosphate buffered saline (PBS) (Thermo Fisher Scientific, 10010072) for 15 minutes at room temperature (RT). Cells were rinsed three times with PBS before proceeding to permeabilization or blocking. For staining of Oct-3/4 and PU.1, cells were permeabilized, while not for cell surface proteins' staining. Permeabilization was conducted with 0.25% Triton-X-100 (Thermo Fisher Scientific, 85111) in 1×PBS for 15 minutes at RT followed by three rinses with PBS. Cells were then blocked with 1% bovine serum albumin (BSA) in PBS for one hour at RT. For primary and secondary antibody staining, antibodies were diluted in PBS with 1% BSA and incubated with cells for one hour at RT. Three 5-minute washes with PBS were used to remove excessive antibodies after staining. Cells were directly imaged in plate on a Nikon Ti2 Eclipse inverted microscope with a Plan Apo Lambda DM 60× (1.4 NA, Ph3) oil objective and an Andor Zyla sCMOS camera. Images were acquired by NIS-Element AR software. All antibodies were used at 1:200 dilution. Primary IF antibodies used in this study were: Oct-3/4 (Santa Cruz Biotechnology, sc-5279), PU.1 (Thermo Fisher Scientific, PA5-17505), CD11b (BioLegend, 101202), P2RY12 (Thermo Fisher Scientific, 702516), CX3CR1 (Abcam, ab8021).
Phagocytosis assay. Differentiated cells were incubated with 20 g/mL of pHrodo Green S. aureus BioParticles (Thermo Fisher Scientific, P35382) for 0-4 hours in mTeSR Plus media in the presence of 100 μg/ml Penicillin-Streptomycin (Corning, 30-002-CI). After removal of excessive particles with PBS washes, cells were harvested for antibody (CX3CR1, P2RY12, CD11b) staining and flow cytometry analysis as described in previous section. Remaining stained cells after flow cytometry was transferred into μ-Plate 96 Well Black plate for fluorescence microscopy to confirm the intracellular localization of the particles. This step needs to be conducted swiftly after flow cytometry in order to avoid changing of cellular morphology due to cell death.
Calcium imaging. Calcium imaging experiment was conducted in standard 12-well cell culture plates. Differentiated cells were incubated with 1 μg/mL Fluo-4 AM calcium indicator (Thermo Fisher Scientific, F23917) in 1 mL of mTeSR Plus media for 30 minutes in a cell culture incubator. Excessive dye was washed away with two 1 mL media washes. After adding 1 mL of fresh mTeSR Plus, the cells were put on stage in a microscope inside the incubator. Images acquisition started without stimulation for 90 seconds to determine baseline signal. One image was acquired every three seconds, the fastest possible on the instrument. After 90 seconds 1 mL of media containing 150 μM ADP was added to the cells while imaging was continuing. The total length of imaging was 10 minutes. Fluorescent signal was quantified and plotted in MATLAB.
Amyloid fibrillation. Aβ fibrillation experiments were performed using SensoLyte Thioflavin T β-Amyloid (1-42) Aggregation Kit (AnaSpec, AS-72214) according to manufacturer's instruction. The reaction was set up in μ-Plate 96 Well Black plate. Data was acquired on a plate reader with excitation/emission=440 nm/484 nm at 37 degree once every 5 minutes for 3 hours.
Preparation of datasets for building single-cell reference atlas. Files containing raw counts of 10× Genomics Chromium scRNA-seq data for different human tissues were download from PanglaoDB (https://panglaodb.se/index.html). Human primary brain single-cell data from gestational weeks 6-22 were downloaded from Organoid Report Card (https://cells.ucsc.edu/?ds=organoidreportcard). Human endometrium single-cell data were download from GEO GSE111976. All sample went through manual cell filtering using Seurat with different filters on number of gene, UMI, and percentage of mitochondria genes. Tissue annotation was compiled through manual curation of each study by checking what tissue/cell types were used. All raw counts table were merged into one sparse matrix which were then used as input for data integration.
Single-cell atlas integration and mapping. Data integration and projection using CSS or Harmony/Symphony was carried out following instructions from the authors on GitHub (https://github.com/quadbiolab/simspec; https://github.com/immunogenomics/harmony; https://github.com/immunogenomics/symphony). Due to the size of the data, these steps were performed on the 02 cluster of Harvard Medical School with at least 180 Gb memory and 8 cores. Most R objects along the pipeline could be saved as standard R files to same time for repeated analysis, except for the UMAP model file, which required saving and loading through the “uwot” package⁷⁴. Code used for integration and projection, together with key reference and annotations files that could be of use for future explorations are shared along this manuscript.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A pluripotent stem cell (PSC) comprising: one or more engineered polynucleotide comprising an open reading frame encoding a SPI1 protein, a FLI1 protein, and a CEBPA protein.

2. The PSC of claim 1, wherein the one or more engineered polynucleotide comprises a first polycistronic polynucleotide that comprises a first open reading frame encoding the SPI1 protein, a second open reading frame encoding the FLI1 protein, and a third open reading frame encoding the CEBPA protein.

3. The PSC of claim 1, wherein the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the FLI1 protein, and a third open reading frame encoding the CEBPA protein.

4. The PSC of claim 1, wherein the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the CEBPA protein, and a third open reading frame encoding the FLI1 protein.

5. The PSC of any one of the preceding claims, wherein the one or more engineered polynucleotide comprises an open reading frame encoding one or more of a MEF2C protein, a CEBPB protein, and a IRF8 protein.

6. The PSC of claim 5, wherein the one or more engineered polynucleotide comprises an open reading frame encoding two or more of a MEF2C protein, a CEBPB protein, and a IRF8 protein.

7. The PSC of claim 6, wherein the one or more engineered polynucleotide comprises an open reading frame encoding a MEF2C protein, a CEBPB protein, and a IRF8 protein.

8. The PSC of claim 7, wherein the one or more engineered polynucleotide comprises a second polycistronic polynucleotide that comprises the following open reading frames, ordered in the 5′ to 3′ direction of the second polycistronic polynucleotide: a first open reading frame encoding the MEF2C protein, a second open reading frame encoding the CEBPB protein, and a third open reading frame encoding the IRF8 protein.

9. The PSC of claim 7, wherein the one or more engineered polynucleotide comprises a second polycistronic polynucleotide that comprises the following open reading frames, ordered in the 5′ to 3′ direction of the second polycistronic polynucleotide: a first open reading frame encoding the MEF2C protein, a second open reading frame encoding the IRF8 protein, and a third open reading frame encoding the CEBPB protein.

10. The PSC of claim 1, wherein the one or more engineered polynucleotide comprises:
a first polycistronic polynucleotide that comprises, optionally 5′ to 3′, a first open reading frame encoding the SPI1 protein, a second open reading frame encoding the CEBPA protein, and a third open reading frame encoding the FLI1 protein; and
a second polycistronic polynucleotide that comprises, optionally 5′ to 3′, a first open reading frame encoding the MEF2C protein, a second open reading frame encoding the CEBPB protein, and a third open reading frame encoding the IRF8 protein.

11. The PSC of any one of the preceding claims, wherein one or more of the open reading frames of the one or more engineered polynucleotide is operably linked to a heterologous promoter.

12. The PSC of claim 11, wherein the heterologous promoter is an inducible promoter.

13. The PSC of claim 12, wherein the inducible promoter is a chemically-inducible promoter.

14. A pluripotent stem cell (PSC) comprising: a SPI1 protein, a FLI1 protein, and a CEBPA protein, wherein the proteins are overexpressed.

15. The PSC of claim 14, wherein the PSC further comprises one or more of a MEF2C protein, a CEBPB protein, and a IRF8 protein, wherein the one or more of the MEF2C protein, the CEBPB protein, and the IRF8 protein is overexpressed.

16. The PSC of claim 15, wherein the PSC further comprises two or more of a MEF2C protein, a CEBPB protein, and a IRF8 protein, wherein the two or more of the MEF2C protein, the CEBPB protein, and the IRF8 protein are overexpressed.

17. The PSC of claim 15, wherein the PSC further comprises a MEF2C protein, a CEBPB protein, and a IRF8 protein, wherein the MEF2C protein, the CEBPB protein, and the IRF8 protein are overexpressed.

18. The PSC of any one of the preceding claims, wherein the PSC is a human PSC.

19. The PSC of any one of the preceding claims, wherein the PSC is an induced PSC (iPSC).

20. A composition comprising: a population of the PSC of any one of the preceding claims.

21. A method, comprising: culturing, in culture media, a population of pluripotent stem cells (PSCs) to produce an expanded population of PSCs; and expressing in PSCs of the expanded population a SPI1 protein, a CEBPA protein, and a FLI1 protein, to produce a population of microglia-like cells.

22. The method of claim 21, wherein the PSCs of the expanded population comprise one or more engineered polynucleotide comprising an open reading frame encoding a SPI1 protein, a FLI1 protein, and a CEBPA protein.

23. The method of claim 22, wherein the one or more engineered polynucleotide comprises a first polycistronic polynucleotide that comprises a first open reading frame encoding the SPI1 protein, a second open reading frame encoding the FLI1 protein, and a third open reading frame encoding the CEBPA protein.

24. The method of claim 22, wherein the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the FLI1 protein, and a third open reading frame encoding the CEBPA protein.

25. The method of claim 22, wherein the one or more engineered polynucleotide comprises a first polycistronic polynucleotide that comprises the following open reading frames, ordered in the 5′ to 3′ direction of the first polycistronic polynucleotide: a first open reading frame encoding the SPI1 protein, a second open reading frame encoding the CEBPA protein, and a third open reading frame encoding the FLI1 protein.

26. The method of any one of the preceding claims, further comprising expressing in PSCs of the expanded population one or more of a MEF2C protein, a CEBPB protein, and a IRF8 protein.

27. The method of any one of claim 22-26, wherein the one or more engineered polynucleotide further comprises an open reading frame encoding one or more of a MEF2C protein, a CEBPB protein, and a IRF8 protein.

28. The method of any one of the preceding claims, further comprising expressing in PSCs of the expanded population two or more of a MEF2C protein, a CEBPB protein, and a IRF8 protein.

29. The method of any one of claim 22-28, wherein the one or more engineered polynucleotide further comprises an open reading frame encoding two or more of a MEF2C protein, a CEBPB protein, and a IRF8 protein.

30. The method of any one of the preceding claims, further comprising expressing in PSCs of the expanded population a MEF2C protein, a CEBPB protein, and a IRF8 protein.

31. The method of any one of claim 22-29, wherein the one or more engineered polynucleotide further comprises an open reading frame encoding a MEF2C protein, a CEBPB protein, and a IRF8 protein.

32. The method of any one of claim 22-29, wherein the one or more engineered polynucleotide further comprises a second polycistronic polynucleotide that comprises the following open reading frames, ordered in the 5′ to 3′ direction of the second polycistronic polynucleotide: a first open reading frame encoding the MEF2C protein, a second open reading frame encoding the CEBPB protein, and a third open reading frame encoding the IRF8 protein.

33. The method of any one of claim 22-29, wherein the one or more engineered polynucleotide further comprises a second polycistronic polynucleotide that comprises the following open reading frames, ordered in the 5′ to 3′ direction of the second polycistronic polynucleotide: a first open reading frame encoding the MEF2C protein, a second open reading frame encoding the IRF8 protein, and a third open reading frame encoding the CEBPB protein.

34. The method of claim 21, wherein the PSCs of the expanded population comprise:
a first polycistronic polynucleotide that comprises, optionally 5′ to 3′, a first open reading frame encoding the SPI1 protein, a second open reading frame encoding the CEBPA protein, and a third open reading frame encoding the FLI1 protein; and
a second polycistronic polynucleotide that comprises, optionally 5′ to 3′, a first open reading frame encoding the MEF2C protein, a second open reading frame encoding the CEBPB protein, and a third open reading frame encoding the IRF8 protein.

35. The method of any one of the preceding claims, wherein one or more of the open reading frames of the one or more engineered polynucleotide is operably linked to a heterologous promoter.

36. The method of claim 35, wherein the heterologous promoter is an inducible promoter.

37. The method of any one of the preceding claims, wherein the population of PSCs comprises 1×10²-1×10⁷ PSCs.

38. The method of any one of the preceding claims, wherein the population of PSCs is cultured for about 2-5 days.

39. The method of claim 38, wherein the population of PSCs is cultured for about 4 days.

40. The method of any one of the preceding claims, wherein microglia-like cells of the population of microglia-like cells are CD11b⁺, CX3CR1⁺, ITGAM⁺, P2RY12⁺, TMEM119⁺, and/or TREM2⁺.

41. The method of any one of the preceding claims, wherein the microglia-like cells of the population of microglia-like cells are TRA-1-60⁻ and/or POU5F1⁻.

42. A method comprising:
(a) delivering to pluripotent stem cells (PSCs) one or more engineered polynucleotide comprising an inducible promoter operably linked to one or more open reading frame encoding a SPI1 protein, a CEBPA protein, and a FLI1 protein, optionally wherein the one or more engineered polynucleotide further comprises an inducible promoter operably linked to one or more open reading frame encoding a MEF2C protein, a CEBPB protein, and/or a IRF8 protein; and
(b) culturing the PSCs of the expanded population in induction media comprising an inducing agent to produce CD11b⁺, CX3CR1⁺, ITGAM⁺, P2RY12⁺, TMEM119⁺, TREM2⁺, TRA-1-60⁻ and/or POU5F1⁻ microglia-like cells.

43. A method comprising:
(a) delivering to pluripotent stem cells (PSCs) one or more engineered polynucleotide comprising an inducible promoter operably linked to one or more open reading frame encoding a SPI1 protein, a CEBPA protein, and a FLI1 protein, optionally wherein the one or more engineered polynucleotide further comprises an inducible promoter operably linked to one or more open reading frame encoding a MEF2C protein, a CEBPB protein, and/or a IRF8 protein;
(b) seeding the PSCs in feeder-free, serum-free culture media and optionally culturing the PSCs for about 1 to about 24 hours; and
(c) culturing the PSCs of (b) in induction media comprising an inducing agent to produce CD11b⁺, CX3CR1⁺, ITGAM⁺, P2RY12⁺, TMEM119⁺, TREM2⁺, TRA-1-60⁻ and/or POU5F1⁻ microglia-like cells.

44. The method of claim 42 or 43 comprising delivering to PSCs (i) a first polycistronic polynucleotide comprising a first inducible promoter operably linked to an open reading frame encoding SPI1, an open reading frame encoding CEBPA, and an open reading frame encoding FLI1, and (ii) a second polycistronic polynucleotide comprising a second inducible promoter operably linked to an open reading frame encoding MEF2C, open reading frame encoding CEBPB, and an open reading frame encoding IRF8.

45. The method of any one of any one of claims 42-44, wherein the first and/or second polycistronic polynucleotide is a transposon and the delivering further comprises delivering a transposase to the PSCs.

46. The method of any one of claims 42-45, wherein the first and/or second inducible promoter is a chemically-inducible promoter, optionally a doxycycline-inducible promoter.

47. The method of any one of claims 42-46, wherein the feeder-free, serum-free culture media of (b) comprises a solubilized basement membrane preparation extracted from the Engelbreth-Holm-Swarm (EHS) mouse sarcoma.

48. The method of claim 47, wherein the solubilized basement membrane preparation comprises extracellular matrix (ECM) proteins and growth factors.

49. The method of claim 48, wherein the ECM proteins are selected from Laminin, Collagen IV, heparan sulfate proteoglycans, and entactin/nidogen.

50. The method of any one of claims 42-49, wherein the feeder-free, serum-free culture media of further comprises growth factors selected from recombinant human basic fibroblast growth factor (rh bFGF) and recombinant human transforming growth factor β (rh TGFβ).

51. The method of any one of claims 42-50, wherein the PSCs of the expanded population of (c) are cultured at a density of about 20,000 cells/cm² to about 60,000 cells/cm².

52. The method of any one of claims 43-51, wherein the feeder-free, serum-free culture media of (b) further comprises a small molecule ROCK inhibitor.

53. The method of any one of claims 42-52, wherein the inducing agent comprises doxycycline.

54. The method of any one of claims 42-53, wherein the culturing the PSCs is an induction media is for about 72 to about 96 hours.

55. A microglia-like cell produced by the method of any one of the preceding claims.
